(19) United States
(12) Patent Application Publication
(10) Pub. No.: US 2007/0292378 A1
(43) Pub. Date: Dec. 20, 2007

(54) Hair-treatment compositions with corneocyte proteins or polypeptides and silicone(s)

(76) Inventors: Petra SOMFLETH, Neu Wulmstorf (DE); Erik Schulze Zur Wiesche, Hamburg (DE); Marcus Krueger, Hamburg (DE)

Correspondence Address: PAUL & PAUL, 2000 MARKET STREET, PHILADELPHIA, PA, 19103-3229, US

(21) Appl. No.: 11/768,089

(22) Filed: Jun. 25, 2007

Related U.S. Application Data
(63) Continuation of application No. PCT/EP05/12424, filed on Nov. 21, 2005.

(30) Foreign Application Priority Data
Dec. 27, 2004 (DE) 10 2004 063 627.3

(51) Int. Cl. A61K 8/89; A61K 8/64

(52) U.S. Cl. 424/70.12; 424/70.14

(57) ABSTRACT

Hair-treatment compositions with advantageous properties comprise 0.01 to 5% by weight of at least one corneocyte protein or polypeptide, and 0.05 to 95% by weight of at least one silicone.

### CROSS-REFERENCE TO RELATED APPLICATIONS

This application is a continuation under 35 U.S.C. § 365 and 35 U.S.C. § 120 of International Application No. PCT/EP2005/012424, filed Nov. 21, 2005. This application also claims priority under 35 U.S.C. § 119 of German Application No. DE 10 2004 063 627.3, filed Dec. 27, 2004.

### STATEMENT REGARDING FEDERALLY SPONSORED RESEARCH OR DEVELOPMENT

Not Applicable

### INCORPORATION-BY-REFERENCE OF MATERIAL SUBMITTED ON A COMPACT DISC

Not Applicable

### BACKGROUND OF THE INVENTION

(1) Field of the Invention

The invention relates to hair-treatment compositions comprising corneocyte proteins or polypeptides and silicone(s), and to the use of these compositions for the cleansing and/or care of skin and hair.

(2) Description of Related Art, Including Information Disclosed Under 37 C.F.R. §§ 1.97 and 1.98.

Not Applicable

The cosmetic treatment of skin and hair is an important constituent of human body care. Thus, human hair is nowadays treated in diverse ways with hair cosmetic preparations. These include, for example, cleansing the hair with shampoos, care and regeneration with rinses and treatments, and bleaching, coloring and shaping the hair with colorants, tints, waving compositions and styling preparations. In this connection, compositions for changing or nuancing the color of head hair play a prominent role. Disregarding bleaching compositions, which bring about oxidative lightening of the hair by degrading the natural hair dyes, essentially three types of hair colorants are of importance in the field of hair coloring.

For permanent, intensive colors with corresponding fastness properties, so-called oxidation colorants are used. Such colorants usually comprise oxidation dye precursors, so-called developer components and coupler components. Under the influence of oxidizing agents or of atmospheric oxygen, the developer components form the actual dyes with one another or by coupling with one or more coupler components. The oxidation colorants are characterized by excellent long-lasting color results. However, for natural-looking colors, it is usually necessary to use a mixture of a relatively large number of oxidation dye precursors; in many cases, direct dyes are also used for the nuancing. If the dyes formed and/or used directly in the course of the color formation have considerably different fastnesses (e.g., UV stability, fastness to perspiration, wash fastness etc.), then a visible and, therefore, undesired color shift may result over time. This phenomenon arises to an increased degree if the hair style has hair or hair zones of differing degree of damage. One example of this is long hair in which the hair ends have for a long time been subjected to all possible environmental influences and are generally considerably more damaged than the relatively freshly regrown hair zones.

For temporary colors, use is usually made of colorants or tints which comprise so-called direct dyes as coloring component. These are dye molecules which attach directly to the hair and require no oxidative process to develop the color. These dyes include, for example, henna, which is known from antiquity for the coloring of body and hair. These colors are generally considerably more sensitive to shampooing than oxidative colors, meaning that an often undesired nuance shift or even a visible “decoloration” arises very much more quickly.

Finally, a new type of coloring method has recently received great attention. In this method, precursors of the natural hair dye melanin are applied to the hair; in the course of oxidative processes within the hair, these then form nature-analogous dyes. In such methods, 5,6-dihydroxyindoline, for example, is used as dye precursor. Upon, in particular, repeated, application of compositions containing 5,6-dihydroxyindoline, it is possible to restore the natural hair color in people with gray hair. The coloration can take place here with atmospheric oxygen as the sole oxidizing agent, meaning that it is not necessary to have recourse to any other oxidizing agents. In the case of people with originally mid-blonde to brown hair, the indoline can be used as the sole dye precursor. For application in the case of people with an originally red and in particular, dark to black hair color, on the other hand, satisfactory results can often only be achieved through co-use of further dye components, in particular, special oxidation dye precursors.

Not least as a result of the considerable stressing of the hair, for example, as a result of coloring or permanent waving or as a result of cleansing the hair with shampoos and as a result of environmental impacts, the importance of care products with as long-lasting an effect as possible is increasing. Such care compositions influence the natural structure and the properties of the hair. Thus, for example, following such treatments, the wet and dry combability of the hair, the hold and the fullness of the hair can be optimized or the hair can be protected against an increased rate of split ends.

It has, therefore, for some time been customary to subject the hair to a special aftertreatment. In this connection, the hair is treated with special active ingredients, for example, quaternary ammonium salts or special polymers, usually in the form of a rinse. Depending on the formulation, this treatment improves the combability, the hold and the fullness of the hair and reduces the rate of split ends.

Furthermore, so-called combination preparations have recently been developed in order to reduce the expenditure of the usual multistage methods, particularly in the case of direct application by consumers.

Besides the customary components, for example, for cleansing the hair, these preparations additionally comprise active ingredients which were previously reserved for hair aftertreatment compositions. The consumer thus saves one application step; at the same time, the packaging expenditure is reduced since one less product is used.

The active ingredients available both for separate aftertreatment compositions and for combination preparations generally act preferably on the surface of the hair. For example, active ingredients are known which impart shine, hold, fullness, better wet or dry combabilities to the hair or prevent split ends. However, just as important as the external appearance of the hair is the internal structural cohesion of the hair fibers, which can be influenced greatly especially during oxidative and reductive processes such as coloring and permanent waving.

However, the known active ingredients can not meet all requirements to an adequate degree. There therefore continues to be a need for active ingredients and active ingredient combinations for cosmetic compositions with good care properties and good biodegradability. Particularly in dye- and/or electrolyte-containing formulations, there is a need for additional care active ingredients which can be incorporated into known formulations without problems.

### BRIEF SUMMARY OF THE INVENTION

It has now been found that particularly advantageous results are achieved if corneocyte proteins or polypeptides in combination with silicone(s) are incorporated into hair-treatment compositions.

### BRIEF DESCRIPTION OF THE SEVERAL VIEWS OF THE DRAWING(S)

Not Applicable

### DETAILED DESCRIPTION OF THE INVENTION

The present invention firstly relates to corneocyte protein- or peptide-containing hair-treatment compositions, comprising

a) 0.01 to 5% by weight of at least one corneocyte protein or polypeptide;

b) 0.05 to 95% by weight of at least one silicone.

The stratum corneum (SC), also referred to as the horny layer, forms the outer layer of the epidermis and serves primarily as a permeability barrier. This protective layer consists of 14 to 27 layers of tightly packed, platelet-like, anuclear, keratin-rich and continually flaking corneocytes (horny cells). The thickness of the stratum corneum varies between 6 and 15 μm, it being pronounced on the palms of the hands and soles of the feet to a significantly greater degree than on other parts of the body.

In principle, the structure of the SC can be described as a two-compartment model. According to the bricks-and-mortar principle, dedifferentiated, protein-containing cells are embedded in a matrix, which can also be viewed as interstitial lipid phase. The cells are responsible for the physical and chemical stability while the intercellular, nonpolar lipids, being a cementing substance, prevent the penetration of water and substances dissolved therein and thus control the water retention and evaporation of water. Insoluble keratin fractions, a hydratable and swellable substance, water and lipids are to be mentioned as main constituents of the horny layer. Of these, the intracellular space contains primarily keratin and lipids. Keratin constitutes approximately 80% of the total mass of the corneocytes. The dense packing of the constituents in the cell leads to high stability, strength and elasticity. The inside of the cell is additionally surrounded by a cornified envelope, comprising loricrin, small, proline-rich proteins, filaggrin, elafin, involucrin and cystatin. The intercellular space contains ceramides (sphingolipids), fatty acids and cholesterol in equimolar fractions. Cholesterol esters, triglycerides, glycosphingolipids and cholesterol sulfate are present in the intercellular spaces in lower concentrations. The presence of the lipids in the corresponding composition and their specific structural organization can be considered essential for fashioning an intact barrier function (e.g., protection against loss of water).

The proteins or polypeptides can be obtained from natural sources or be produced by recombinant methods. The proteins or polypeptides can be optionally branched, e.g., by a chemical branching agent or by an enzyme which forms bonds between adjacent polypeptides.

If desired, the proteins or polypeptides can be modified. Such modifications include, for example, chemical derivatization of one or more amino acids or modification of the amino acid sequence of the protein or polypeptide. These modifications can be used in order to impart certain properties to the proteins or polypeptides, such as, for example, higher solubility in water, higher stability against the effect of chemicals, atmospheric or enzymatic influences, etc.

Depending on the molar mass of the amino acids present in the corneocyte proteins or polypeptides, these proteins or polypeptides can contain a few hundred to thousand amino acids.

In addition, preference is also given to hair-treatment compositions according to the invention which comprise at least one corneocyte protein or polypeptide with a molar mass of from 2 to 8 kDa, preferably from 2.5 to 7 kDa, particularly preferably from 2.75 to 6 kDa and in particular, from 3 to 4.5 kDa.

Preference is also given to compositions according to the invention which include both corneocyte proteins and polypeptides of relatively low molecular mass and also those of relatively high molecular mass. Here, preference is given to hair-treatment compositions according to the invention which comprise the corneocyte protein or polypeptide with a molar mass of from 20 to 80 kDa and the corneocyte protein or polypeptide with a molar mass of from 2 to 8 kDa in the weight ratio from 100:1 to 1:100, preferably from 10:1 to 1:10 and in particular, from 5:1 to 1:2.

The compositions according to the invention can comprise further active ingredients and auxiliaries. These are described below.

The use of surfactants (E) in the compositions according to the invention has proven particularly advantageous. In a further preferred embodiment, the compositions according to the invention therefore comprise surfactants. The term surfactants is understood as meaning interface-active substances which can form adsorption layers at surfaces and interfaces or can aggregate in volume phases to give micelle colloids or lyotropic mesophases. A distinction is made between anionic surfactants consisting of a hydrophobic radical and a negatively charged hydrophilic head group, amphoteric surfactants, which carry both a negative and a compensating positive charge, cationic surfactants, which have a positively charged hydrophilic group besides a hydrophobic radical, and nonionic surfactants, which have no charges but strong dipole moments and are highly hydrated in aqueous solution.

Suitable anionic surfactants (E1) in the preparations according to the invention are all anionic surface-active substances suitable for use on the human body. These are characterized by a water-solubilizing, anionic group such as, for example, a carboxylate group, sulfate group, sulfonate group or phosphate group, and a lipophilic alkyl group having about 8 to 30 carbon atoms. In addition, glycol or polyglycol ether groups, ester groups, ether groups and amide groups, and hydroxyl groups may be present in the molecule. Examples of suitable anionic surfactants are, in each case in the form of the sodium, potassium and ammonium salts, and the mono-, di- and trialkanolammonium salts having 2 to 4 carbon atoms in the alkanol group,
linear and branched fatty acids having 8 to 30 carbon atoms (soaps),
ether carboxylic acids of the formula
R—O—(CH₂—CH₂O)ₓ—CH₂—COOH, in which R is a linear alkyl group having 8 to 30 carbon atoms and x is 0 or 1 to 16,
acyl sarcosides having 8 to 24 carbon atoms in the acyl group,
acyl taurides having 8 to 24 carbon atoms in the acyl group,
acyl isethionates having 8 to 24 carbon atoms in the acyl group,
sulfosuccinic mono- and dialkyl esters having 8 to 24 carbon atoms in the alkyl group and sulfosuccinic acid monoalkylpolyoxyethyl esters having 8 to 24 carbon atoms in the alkyl group and 1 to 6 oxyethyl groups,
linear alkanesulfonates having 8 to 24 carbon atoms,
linear alpha-olefinsulfonates having 8 to 24 carbon atoms,
alpha-sulfo fatty acid methyl esters of fatty acids having 8 to 30 carbon atoms,
alkyl sulfates and alkyl polyglycol ether sulfates of the formula R—O(CH₂—CH₂O)ₓ—OSO₃H, in which R is a preferably linear alkyl group having 8 to 30 carbon atoms and x is 0 or 1 to 12,
sulfated hydroxyalkyl polyethylene and/or hydroxyalkylene propylene glycol ethers
sulfonates of unsaturated fatty acids having 8 to 24 carbon atoms and 1 to 6 double bonds,
esters of tartaric acid and citric acid with alcohols, which are addition products of about 2-15 molecules of ethylene oxide and/or propylene oxide onto fatty alcohols having 8 to 22 carbon atoms,
alkyl and/or alkenyl ether phosphates of the formula (E1-I)
   R¹(OCH₂CH₂)ₙ—O—P(O)(OX)—OR² (E1-I)
in which R¹ is preferably an aliphatic hydrocarbon radical having 8 to 30 carbon atoms, R² is hydrogen, a radical (CH₂CH₂O)ₙR² or X, n is numbers from 1 to 10 and X is hydrogen, an alkali metal or alkaline earth metal or NR³R⁴R⁵R⁵, where R³ to R⁶, independently of one another, are hydrogen or a C₁ to C₄ hydrocarbon radical, sulfated fatty acid alkylene glycol esters of the formula (E1-II)
   R⁷CO(AlkO)ₙSO₃M (E1-II)
in which R⁷CO— is a linear or branched, aliphatic, saturated and/or unsaturated acyl radical having 6 to 22 carbon atoms, Alk is CH₂CH₂, CHCH₃CH₂ and/or CH₂CHCH₃, n is numbers from 0.5 to 5 and M is a cation,
monoglyceride sulfates and monoglyceride ether sulfates of the formula (E1-II) 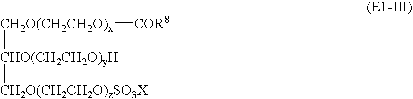
   in which R⁸CO is a linear or branched acyl radical having 6 to 22 carbon atoms, x, y and z are in total 0 or numbers from 1 to 30, preferably 2 to 10, and X is an alkali metal or alkaline earth metal. Typical examples of monoglyceride (ether) sulfates suitable for the purposes of the invention are the reaction products of lauric acid monoglyceride, coconut fatty acid monoglyceride, palmitic acid monoglyceride, stearic acid monoglyceride, oleic acid monoglyceride and tallow fatty acid monoglyceride, and ethylene oxide adducts thereof with sulfur trioxide or chlorosulfonic acid in the form of their sodium salts. Preference is given to using monoglyceride sulfates of the formula (E1-III) in which R⁸CO is a linear acyl radical having 8 to 18 carbon atoms,
amide ether carboxylic acids,
condensation products of C₈-C₃₀-fatty alcohols with protein hydrolyzates and/or amino acids and derivatives thereof, which are known to the person skilled in the art as protein fatty acid condensates, such as, for example, the Lamepon® grades, Gluadin® grades, Hostapon® KCG or the Amisoft® grades.

Preferred anionic surfactants are alkyl sulfates, alkyl polyglycol ether sulfates and ether carboxylic acids having 10 to 18 carbon atoms in the alkyl group and up to 12 glycol ether groups in the molecule, sulfosuccinic acid mono- and dialkyl esters having 8 to 18 carbon atoms in the alkyl group and sulfosuccinic acid monoalkylpolyoxyethyl esters having 8 to 18 carbon atoms in the alkyl group and 1 to 6 oxyethyl groups, monoglyceride sulfates, alkyl and alkenyl ether phosphates, and protein fatty acid condensates.

Zwitterionic surfactants (E2) is the term used to refer to those surface-active compounds which carry at least one quaternary ammonium group and at least one —COO⁽⁻⁾ or —SO₃⁽⁻⁾ group in the molecule. Particularly suitable zwitterionic surfactants are the so-called betaines, such as the N-alkyl-N,N-dimethylammonium glycinates, for example, cocoalkyldimethylammonium glycinate, N-acylaminopropyl-N,N-dimethylammonium glycinates, for example, cocoacylaminopropyldimethylammonium glycinate, and 2-alkyl-3-carboxymethyl-3-hydroxyethylimidazolines having in each case 8 to 18 carbon atoms in the alkyl or acyl group, and cocoacylaminoethyl hydroxyethylcarboxymethyl glycinate. A preferred zwitterionic surfactant is the fatty acid amide derivative known under the INCI name Cocamidopropyl Betaine.

Ampholytic surfactants (E3) are understood as meaning those surface-active compounds which, apart from a C₈-C₂₄-alkyl or -acyl group in the molecule, comprise at least one free amino group and at least one —COOH or —SO₃H group and are capable of forming internal salts. Examples of suitable ampholytic surfactants are N-alkylglycines, N-alkylpropionic acids, N-alkylaminobutyric acids, N-alkyliminodipropionic acids, N-hydroxyethyl-N-alkylamidopropylglycines, N-alkyltaurines, N-alkylsarcosines, 2-alkylamino-propionic acids and alkylaminoacetic acids having in each case about 8 to 24 carbon atoms in the alkyl group. Particularly preferred ampholytic surfactants are N-cocoalkylaminopropionate, cocoacylaminoethylaminopropionate and C₁₂-C₁₈-acylsarcosine.

Nonionic surfactants (E4) comprise, as hydrophilic group, e.g., a polyol group, a polyalkylene glycol ether group or a combination of polyol and polyglycol ether group. Such compounds are, for example,
addition products of from 2 to 50 mol of ethylene oxide and/or 0 to 5 mol of propylene oxide onto linear and branched fatty alcohols having 8 to 30 carbon atoms, onto fatty acids having 8 to 30 carbon atoms and onto alkylphenols having 8 to 15 carbon atoms in the alkyl group,
addition products, terminally capped with a methyl or C₂-C₆-alkyl radical, of from 2 to 50 mol of ethylene oxide and/or 0 to 5 mol of propylene oxide onto linear and branched fatty alcohols having 8 to 30 carbon atoms, onto fatty acids having 8 to 30 carbon atoms and onto alkylphenols having 8 to 15 carbon atoms in the alkyl group, such as, for example, the grades available under the brand names Dehydrol® LS, Dehydrol® LT (Cognis),
C₁₂-C₃₀-fatty acid mono- and diesters of addition products of from 1 to 30 mol of ethylene oxide onto glycerol,
addition products of from 5 to 60 mol of ethylene oxide onto castor oil and hydrogenated castor oil,
polyol fatty acid esters, such as, for example, the commercial product Hydagen® HSP (Cognis) or Sovermol grades (Cognis),
alkoxylated triglycerides,
alkoxylated fatty acid alkyl esters of the formula (E4-I)
   R¹CO—(OCH₂CHR²)_{w}OR³ (E4-I)
in which R¹CO is a linear or branched, saturated and/or unsaturated acyl radical having 6 to 22 carbon atoms, R² is hydrogen or methyl, R³ is linear or branched alkyl radicals having 1 to 4 carbon atoms and w is numbers from 1 to 20,
amine oxides,
hydroxy mixed ethers,
sorbitan fatty acid esters and addition products of ethylene oxide onto sorbitan fatty acid esters, such as, for example, the polysorbates,
sugar fatty acid esters and addition products of ethylene oxide onto sugar fatty acid esters,
addition products of ethylene oxide onto fatty acid alkanolamides and fatty amines,
sugar surfactants of the type of the alkyl and alkenyl oligoglycosides according to formula (E4-II),
   R⁴O-[G]ₚ (E4-II)
in which R⁴ is an alkyl or alkenyl radical having 4 to 22 carbon atoms, G is a sugar radical having 5 or 6 carbon atoms and p is numbers from 1 to 10. They can be obtained by the relevant methods of preparative organic chemistry. The alkyl and alkenyl oligoglycosides can be derived from aldoses or ketoses having 5 or 6 carbon atoms, preferably from glucose. The preferred alkyl and/or alkenyl oligoglycosides are thus alkyl and/or alkenyl oligoglucosides. The index number p in the general formula (E4-II) indicates the degree of oligomerization (DP), i.e., the distribution of mono- and oligoglycosides, and is a number between 1 and 10. While p in the individual molecule must always be an integer and here can in particular, assume values p=1 to 6, the value p for a specific alkyl oligoglycoside is an analytically determined calculated parameter which in most cases is a fraction. Preference is given to using alkyl and/or alkenyl oligoglycosides with an average degree of oligomerization p of from 1.1 to 3.0. From an applications point of view, preference is given to those alkyl and/or alkenyl oligoglycosides whose degree of oligomerization is less than 1.7 and in particular, is between 1.2 and 1.4. The alkyl or alkenyl radical R⁴ can be derived from primary alcohols having 4 to 11, preferably 8 to 10, carbon atoms. Typical examples are butanol, caproic alcohol, capryl alcohol, capric alcohol and undecyl alcohol, and technical-grade mixtures thereof, as are obtained, for example, during the hydrogenation of technical-grade fatty acid methyl esters or in the course of the hydrogenation of aldehydes from the Roelen's oxo synthesis. Preference is given to alkyl oligoglucosides of chain length C₈-C₁₀ (DP=1 to 3), which are produced as forerunning in the distillative separation of technical-grade C₈-C₁₈ coconut fatty alcohol and may be contaminated with a fraction of less than 6% by weight of the C₁₂-alcohol, and alkyl oligoglucosides based on technical-grade C_{9/11}-oxo alcohols (DP=1 to 3). In addition, the alkyl or alkenyl radical R¹⁵ can also be derived from primary alcohols having 12 to 22, preferably 12 to 14, carbon atoms. Typical examples are lauryl alcohol, myristyl alcohol, cetyl alcohol, palmoleyl alcohol, stearyl alcohol, isostearyl alcohol, oleyl alcohol, elaidyl alcohol, petroselinyl alcohol, arachyl alcohol, gadoleyl alcohol, behenyl alcohol, erucyl alcohol, brassidyl alcohol, and technical-grade mixtures thereof, which can be obtained as described above. Preference is given to alkyl oligoglucosides based on hydrogenated C_{12/14} coconut alcohol with a DP of from 1 to 3.
sugar surfactants of the type of fatty acid-N-alkylpolyhydroxyalkylamides, a nonionic surfactant of the formula (E4-III),
   R⁵CO—NR⁶—[Z] (E4-III)
in which R⁵CO is an aliphatic acyl radical having 6 to 22 carbon atoms, R⁶ is hydrogen, an alkyl or hydroxyalkyl radical having 1 to 4 carbon atoms and [Z] is a linear or branched polyhydroxyalkyl radical having 3 to 12 carbon atoms and 3 to 10 hydroxyl groups. The fatty acid-N-alkylpolyhydroxyalkylamides are known substances which are usually obtained by reductive amination of a reducing sugar with ammonia, an alkylamine or an alkanolamine and subsequent acylation with a fatty acid, a fatty acid alkyl ester or a fatty acid chloride. Preferably, the fatty acid-N-alkyl-polyhydroxyalkylamides are derived from reducing sugars having 5 or 6 carbon atoms, in particular, from glucose. The preferred fatty acid-N-alkylpolyhydroxyalkylamides are therefore fatty acid-N-alkylglucamides, as given by the formula (E4-IV):
   R⁷CO—NR⁸—CH₂—(CHOH)₄—CH₂OH (E4-IV)
   As fatty acid-N-alkylpolyhydroxyalkylamides, preference is given to using glucamides of the formula (E4-IV) in which R⁸ is hydrogen or an alkyl group and R⁷CO is an acyl radical of caproic acid, caprylic acid, capric acid, lauric acid, myristic acid, palmitic acid, palmoleic acid, stearic acid, isostearic acid, oleic acid, elaidic acid, petroselinic acid, linoleic acid, linolenic acid, arachidic acid, gadoleic acid, behenic acid or erucic acid or technical-grade mixtures thereof. Particular preference is given to fatty acid-N-alkylglucamides of the formula (E4-IV) which are obtained by reductive amination of glucose with methylamine and subsequent acylation with lauric acid or C12/14 coconut fatty acid or a corresponding derivative. In addition, the polyhydroxyalkylamides can also be derived from maltose and palatinose.

Preferred nonionic surfactants have proven to be the alkylene oxide addition products onto saturated linear fatty alcohols and fatty acids having in each case 2 to 30 mol of ethylene oxide per mole of fatty alcohol or fatty acid. Preparations with excellent properties are likewise obtained if they comprise fatty acid esters of ethoxylated glycerol as nonionic surfactants.

These compounds are characterized by the following parameters. The alkyl radical R comprises 6 to 22 carbon atoms and may either be linear or branched. Preference is given to primary linear and 2-position methyl-branched aliphatic radicals. Such alkyl radicals are, for example, 1-octyl, 1-decyl, 1-lauryl, 1-myristyl, 1-cetyl and 1-stearyl. Particular preference is given to 1-octyl, 1-decyl, 1-lauryl, 1-myristyl. When using so-called “oxo alcohols” as starting materials, compounds with an uneven number of carbon atoms in the alkyl chain predominate.

In addition, very particularly preferred nonionic surfactants are the sugar surfactants. These can be present in the compositions used according to the invention preferably in amounts of 0.1-20% by weight, based on the total composition. Amounts of 0.5-15% by weight are preferred, and very particular preference is given to amounts of 0.5-7.5% by weight.

The compounds with alkyl groups used as surfactant may each be uniform substances. However, it is generally preferred, when producing these substances, to start from native vegetable or animal raw materials, thus giving mixtures of substances with different alkyl chain lengths that are dependent on the respective raw material.

In the case of the surfactants which constitute addition products of ethylene oxide and/or propylene oxide onto fatty alcohols or derivatives of these addition products, it is possible to use either products with a “normal” homolog distribution or those with a narrowed homolog distribution. “Normal” homolog distribution is understood here as meaning mixtures of homologs which are obtained in the reaction of fatty alcohol and alkylene oxide using alkali metals, alkali metal hydroxides or alkali metal alkoxides as catalysts. Narrowed homolog distributions, on the other hand, are obtained if, for example, hydrotalcites, alkaline earth metal salts of ethercarboxylic acids, alkaline earth metal oxides, hydroxides or alkoxides are used as catalysts. The use of products with a narrowed homolog distribution may be preferred.

According to the invention, cationic surfactants of the quaternary ammonium compound type, the ester quat type and the amidoamine type can be used. Preferred quaternary ammonium compounds are ammonium halides, in particular, chlorides and bromides, such as alkyltrimethylammonium chlorides, dialkyldimethylammonium chlorides and trialkylmethylammonium chlorides, e.g., cetyltrimethylammonium chloride, stearyltrimethylammonium chloride, distearyldimethylammonium chloride, lauryldimethylammonium chloride, lauryldimethylbenzylammonium chloride and tricetylmethylammonium chloride, and the imidazolium compounds known under the INCI names Quaternium-27 and Quaternium-83. The long alkyl chains of the above-mentioned surfactants preferably have 10 to 18 carbon atoms.

According to the invention, preference is given to using QAV with behenyl radicals, in particular, the substances known under the name behentrimmonium chloride or bromide (docosanyltrimethylammonium chloride or bromide). Other preferred QAVs have at least two behenyl radicals, where QAV which two behenyl radicals on an imidazolinium backbone are particularly preferred. These substances are commercially available, for example, under the names Genamin® KDMP (Clariant) and Crodazosoft® DBQ (Crodauza).

Ester quats are known substances which contain both at least one ester function and also at least one quaternary ammonium group as structural element. Preferred ester quats are quaternized ester salts of fatty acids with triethanolamine, quaternized ester salts of fatty acids with diethanolalkylamines and quaternized ester salts of fatty acids with 1,2-dihydroxypropyldialkylamines. Such products are sold, for example, under the trade names Stepantex®, Dehyquart® and Armocare®. The products Armocare® VGH-70, an N,N-bis(2-palmitoyloxyethyl)dimethylammonium chloride, and Dehyquart® F-75, Dehyquart® C-4046, Dehyquart® L80 and Dehyquart® AU-35 are examples of such ester quats.

The alkylamidoamines are usually prepared by amidation of natural or synthetic fatty acids and fatty acid cuts with dialkylaminoamines. One compound from this group of substances which is particularly suitable according to the invention is the stearamidopropyldimethylamine commercially available under the name Tegoamid® S 18.

The cationic surfactants are present in the compositions according to the invention preferably in amounts of from 0.05 to 10% by weight, based on the total composition. Amounts of from 0.1 to 5% by weight are particularly preferred. Within the scope of the present invention, “based on the composition” means here “based on the mixture of the preparation of oxidation dye precursors (A) and the oxidizing agent preparation (B)”.

The surfactants (E) are used in amounts of from 0.1-45% by weight, preferably 0.5-30% by weight and very particularly preferably from 0.5-25% by weight, based on the total composition used according to the invention.

Anionic, nonionic, zwitterionic and/or amphoteric surfactants, and mixtures thereof, may be preferred according to the invention.

In summary, preference is given to hair-treatment compositions according to the invention which comprise—based on their weight—0.5 to 70% by weight, preferably 1 to 60% by weight and in particular, 5 to 25% by weight, of anionic and/or nonionic and/or cationic and/or amphoteric surfactant(s).

A further preferred group of ingredients of the hair-treatment compositions according to the invention are vitamins, provitamins or vitamin precursors. These are described below.

Hair-treatment composition as claimed in one of claims 1 to 13, which comprises vitamins, provitamins and vitamin precursors which are assigned to the groups A, B, C, E, F and H, where preferred compositions comprise the specified compounds in amounts of from 0.1 to 5% by weight, preferably from 0.25 to 4% by weight and in particular, from 0.5 to 2.5% by weight, in each case based on the total composition.

The group of the substances referred to as vitamin A includes retinol (vitamin A₁) and 3,4-didehydroretinol (vitamin A₂). β-Carotene is the provitamin of retinol. Suitable as vitamin A component are, according to the invention, for example, vitamin A acid and esters thereof, vitamin A aldehyde and vitamin A alcohol, and esters thereof, such as the palmitate and the acetate. The compositions according to the invention comprise the vitamin A component preferably in amounts of 0.05-1% by weight, based on the total preparation.

The vitamin B group or the vitamin B complex includes, inter alia,
vitamin B₁ (thiamine)
vitamin B₂ (riboflavin)
vitamin B₃. This name often covers the compounds nicotinic acid and nicotinamide (niacinamide). According to the invention, preference is given to nicotinamide, which is present in the compositions used according to the invention preferably in amounts of from 0.05 to 1% by weight, based on the total composition.
Vitamin B₅ (pantothenic acid, panthenol and pantolactone). Within the scope of this group, preference is given to using panthenol and/or pantolactone. Derivatives of the panthenol which can be used according to the invention are, in particular, the esters and ethers of panthenol, and cationically derivatized panthenols. Individual representatives are, for example, panthenol triacetate, panthenol monoethyl ether and its monoacetate, and the cationic panthenol derivatives disclosed in WO 92/13829. The specified compounds of the vitamin B₅ type are present in the compositions used according to the invention preferably in amounts of 0.05-10% by weight, based on the total composition. Amounts of 0.1-5% by weight are particularly preferred.
Vitamin B₆ (pyridoxine and pyridoxamine and pyridoxal).
Vitamin C (ascorbic acid). Vitamin C is used in the compositions according to the invention preferably in amounts of from 0.1 to 3% by weight, based on the total composition. Use in the form of the palmitic acid ester, the glucosides or phosphates may be preferred. Use in combination with tocopherols may likewise be preferred

Vitamin E (tocopherols, in particular, α-tocopherol). Tocopherol and its derivatives, which include, in particular, the esters, such as the acetate, the nicotinate, the phosphate and the succinate, are present in the compositions used according to the invention preferably in amounts of 0.05-1% by weight, based on the total composition.

Vitamin F. The term “vitamin F” is usually understood as meaning essential fatty acids, in particular, linoleic acid, linolenic acid and arachidonic acid.

Vitamin H. Vitamin H is the term used to refer to the compound (3aS,4S,6aR)-2-oxohexahydrothienol[3,4-d]imidazole-4-valeric acid, for which, however, the trivial name biotin has meanwhile caught on. Biotin is present in the compositions used according to the invention preferably in amounts of from 0.0001 to 1.0% by weight, in particular, in amounts of from 0.001 to 0.01% by weight.

In summary, preference is given to hair-treatment compositions according to the invention which comprise vitamins, provitamins and vitamin precursors which are assigned to the groups A, B, C, E, F and H, where preferred compositions comprise the specified compounds in amounts of from 0.1 to 5% by weight, preferably from 0.25 to 4% by weight and in particular, from 0.5 to 2.5% by weight, in each case based on the total composition.

The hair-treatment compositions according to the invention comprise silicone(s) in amounts of from 0.05 to 95% by weight, in each case based on the total composition. In this connection, only special products such as, for example, hair end fluids, usually comprise large amounts of silicones, whereas other products such as shampoos, conditioners, hair treatments etc. more likely comprise amounts below 20% by weight. Depending on the nature of the composition according to the invention, the contents of silicone(s) are therefore entirely variable. If the compositions according to the invention are formulated as shampoos, particularly preferred hair-treatment compositions according to the invention are those which comprise silicone(s) in amounts of from 0.1 to 10% by weight, preferably from 0.25 to 5% by weight and in particular, from 0.5 to 2.5% by weight, in each case based on the total composition.

Particular preference is given to hair-treatment compositions according to the invention which comprise at least one silicone selected from:
(i) polyalkylsiloxanes, polyarylsiloxanes, polyalkylarylsiloxanes, which are volatile or nonvolatile, straight-chain, branched or cyclic, crosslinked or uncrosslinked;
(ii) polysiloxanes which contain one or more organofunctional groups in their general structure which are selected from:
   a) substituted or unsubstituted aminated groups;
   b) (per)fluorinated groups;
   c) thiol groups;
   d) carboxylate groups;
   e) hydroxylated groups;
   f) alkoxylated groups;
   g) acyloxyalkyl groups;
   h) amphoteric groups;
   i) bisulfite groups;
   j) hydroxyacylamino groups;
   k) carboxy groups;
   l) sulfonic acid groups; and
   m) sulfate or thiosulfate groups;
(iii) linear polysiloxane(A)-polyoxyalkylene(B) block copolymers of the type (A-B)ₙ where n>3;
(iv) grafted silicone polymers with non-silicone-containing, organic basic backbone which consist of an organic main chain which is formed from organic monomers which do not contain silicone, onto which at least one polysiloxane macromer has been grafted in the chain and optionally on at least one chain end;
(v) grafted silicone polymers with polysiloxane backbone, onto which non-silicon-containing, organic monomers have been grafted which have a polysiloxane main chain, onto which at least one organic macromer which does not contain silicone has been grafted in the chain and optionally on at least one of its ends;
(vi) or mixtures thereof.

Particularly preferred hair-treatment compositions according to the invention are characterized in that they comprise at least one silicone of the formula I
(CH₃)₃Si—[O—Si(CH₃)₂]ₓ—O—Si(CH₃)₃ (I),
in which x is a number from 0 to 100, preferably from 0 to 50, further preferably from 0 to 20 and in particular, 0 to 10.

The hair-treatment compositions preferred according to the invention comprise a silicone of the above formula I. These silicones are referred to as DIMETHICONES according to INCI nomenclature. For the purposes of the present invention, the silicone of the formula I used is preferably the compounds:
(CH₃)₃Si—O—Si(CH₃)₃
(CH₃)₃Si—O—(CH₃)₂Si—O—Si(CH₃)₃
(CH₃)₃Si—[O—(CH₃)₂Si]₂—O—Si(CH₃)₃
(CH₃)₃Si—[O—(CH₃)₂Si]₃—O—Si(CH₃)₃
(CH₃)₃Si—[O—(CH₃)₂Si]₄—O—Si(CH₃)₃
(CH₃)₃Si—[O—(CH₃)₂Si]₅—O—Si(CH₃)₃
(CH₃)₃Si—[O—(CH₃)₂Si]₆—O—Si(CH₃)₃
(CH₃)₃Si—[O—(CH₃)₂Si]₇—O—Si(CH₃)₃
(CH₃)₃Si—[O—(CH₃)₂Si]₈—O—Si(CH₃)₃
(CH₃)₃Si—[O—(CH₃)₂Si]₉—O—Si(CH₃)₃
(CH₃)₃Si—[O—(CH₃)₂Si]₁₀—O—Si(CH₃)₃
(CH₃)₃Si—[O—(CH₃)₂Si]₁₁—O—Si(CH₃)₃
(CH₃)₃Si—[O—(CH₃)₂Si]₁₂—O—Si(CH₃)₃
(CH₃)₃Si—[O—(CH₃)₂Si]₁₃—O—Si(CH₃)₃
(CH₃)₃Si—[O—(CH₃)₂Si]₁₄—O—Si(CH₃)₃
(CH₃)₃Si—[O—(CH₃)₂Si]₁₅—O—Si(CH₃)₃
(CH₃)₃Si—[O—(CH₃)₂Si]₁₆—O—Si(CH₃)₃
(CH₃)₃Si—[O—(CH₃)₂Si]₁₇—O—Si(CH₃)₃
(CH₃)₃Si—[O—(CH₃)₂Si]₁₈—O—Si(CH₃)₃
(CH₃)₃Si—[O—(CH₃)₂Si]₁₉—O—Si(CH₃)₃
(CH₃)₃Si—[O—(CH₃)₂Si]₂₀—O—Si(CH₃)₃
   where (CH₃)₃Si—O—Si(CH₃)₃, (CH₃)₃Si—O—(CH₃)₂Si—O—Si(CH₃)₃ and/or (CH₃)₃Si—[O—(CH₃)₂Si]₂—O—Si(CH₃)₃ are particularly preferred.

Mixtures of the abovementioned silicones may of course also be present in the compositions according to the invention.

Preferred silicones which can be used according to the invention have, at 20° C., viscosities of from 0.2 to 2 mm²s⁻¹, where silicones with viscosities of from 0.5 to 1 mm²s⁻¹ are particularly preferred.

Particularly preferred compositions according to the invention comprise one or more aminofunctional silicones. Such silicones can, for example, be described by the formula
M(RₐQ_{b}SiO_{(4-a-b)/2)x}(R_{c}SiO_{(4-c)/2)y}M
where, in the above formula, R is a hydrocarbon or a hydrocarbon radical having 1 to about 6 carbon atoms, Q is a polar radical of the general formula —R¹HZ, in which R¹ is a divalent, linking group which is bonded to hydrogen and the radical Z, composed of carbon and hydrogen atoms, carbon, hydrogen and oxygen atoms or carbon, hydrogen and nitrogen atoms, and Z is an organic, amino-functional radical which contains at least one aminofunctional group; “a” assumes values in the range from about 0 to about 2, “b” assumes values in the range from about 1 to about 3, “a”+“b” is less than or equal to 3, and “c” is a number in the range from about 1 to about 3, and x is a number in the range from 1 to about 2,000, preferably from about 3 to about 50 and most preferably from about 3 to about 25, and y is a number in the range from about 20 to about 10,000, preferably from about 125 to about 10 000 and most preferably from about 150 to about 1,000, and M is a suitable silicone end group, as is known in the prior art, preferably trimethylsiloxy. Nonlimiting examples of the radicals represented by R include alkyl radicals, such as methyl, ethyl, propyl, isopropyl, isopropyl, butyl, isobutyl, amyl, isoamyl, hexyl, isohexyl and the like; alkenyl radicals, such as vinyl, halovinyl, alkylvinyl, allyl, haloallyl, alkylallyl; cycloalkyl radicals, such as cyclobutyl, cyclopentyl, cyclohexyl and the like; phenyl radicals, benzyl radicals, halogenated hydrocarbon radicals, such as 3-chloropropyl, 4-bromobutyl, 3,3,3-tri-fluoropropyl, chlorocyclohexyl, bromophenyl, chlorophenyl and the like, and sulfur-containing radicals, such as mercaptoethyl, mercaptopropyl, mercaptohexyl, mercaptophenyl and the like; preferably, R is an alkyl radical which contains 1 to about 6 carbon atoms, and most preferably R is methyl. Examples of R¹ include methylene, ethylene, propylene, hexa-methylene, decamethylene, —CH₂CH(CH₃)CH₂—, phenylene, naphthylene, —CH₂CH₂SCH₂CH₂—, —CH₂CH₂OCH₂—, —OCH₂CH₂—, —OCH₂CH₂CH₂—, —CH₂CH(CH₃)C(O)OCH₂—, —(CH₂)₃CC(O)OCH₂CH₂—, —C₆H₄C₆H₄—, —C₆H₄CH₂C₆H₄—; and —(CH₂)₃C(O)SCH₂CH₂—.

Z is an organic aminofunctional radical comprising at least one functional amino group. One possible formula for Z is NH(CH₂)_{z}NH₂, in which z is 1 or more. Another possible formula for Z is —NH(CH₂)_{z}(CH₂)_{zz}NH, in which both z and zz, independently, are 1 or more, where this structure includes diamino ring structures, such as piperazinyl. Z is most preferably a —NHCH₂CH₂NH₂ radical. Another possible formula for Z is —N(CH₂)_{z}(CH₂)_{zz}NX₂ or —NX₂, in which each X is selected independently of X₂ from the group consisting of hydrogen and alkyl groups having 1 to 12 carbon atoms, and zz is 0.

Q is most preferably a polar, aminofunctional radical of the formula —CH₂CH₂CH₂NHCH₂CH₂NH₂. In the formulas, “a” assumes values in the range from about 0 to about 2, “b” assumes values in the range from about 2 to about 3, “a”+“b” is less than or equal to 3, and “c” is a number in the range from about 1 to about 3. The molar ratio of RₐQ_{b}SiO_{(4-a-b)/2} units to the R_{c}SiO_{(4-c)/2} units is in the range from about 1:2 to 1:65, preferably from about 1:5 to about 1:65 and most preferably from about 1:15 to about 1:20. If one or more silicones of the above formula are used, the various variable substituents in the above formula can be different for the various silicone components which are present in the silicone mixture.

Preferred hair-treatment compositions according to the invention are characterized in that they comprise an aminofunctional silicone of the formula (II)
R′ₐG₃₋ₐ-Si(OSiG₂)ₙ-(OSiG_{b}R′_{2-b})ₘ—O—SiG₃₋ₐ-R′ₐ (II),
in which:
G is —H, a phenyl group, —OH, —O—CH₃, —CH₃, —O—CH₂CH₃, —CH₂CH₃, —O—CH₂CH₂CH₃, —CH₂CH₂CH₃, —O—CH(CH₃)₂, —CH(CH₃)₂, —O—CH₂CH₂CH₂CH₃, —CH₂CH₂CH₂CH₃, —O—CH₂CH(CH₃)₂, —CH₂CH(CH₃)₂, —O—CH(CH₃)CH₂CH₃, —CH(CH₃)CH₂CH₃, —O—C(CH₃)₃, —C(CH₃)₃;
a is a number between 0 and 3, in particular, 0;
b is a number between 0 and 1, in particular, 1,
m and n are numbers whose sum (m+n) is between 1 and 2,000, preferably between 50 and 150, where n preferably assumes values from 0 to 1,999 and in particular, from 49 to 149 and m preferably assumes values from 1 to 2,000, in particular, from 1 to 10,
R′ is a monovalent radical selected from
   -Q-N(R″)—CH₂—CH₂—N(R″)₂
   -Q-N(R″)₂
   -Q-N⁺(R″)₃A⁻
   -Q-N⁺H(R″)₂A⁻
   -Q-N⁺H₂(R″)A⁻
   -Q-N(R″)—CH₂—CH₂—N⁺R″H₂A⁻,
where each Q is a chemical bond, —CH₂—, —CH₂—CH₂—, —CH₂CH₂CH₂—, —C(CH₃)₂—, —CH₂CH₂CH₂CH₂—, —CH₂C(CH₃)₂—, —CH(CH₃)CH₂CH₂—,
—R″ is identical or different radicals from the group —H, -phenyl, -benzyl, —CH₂—CH(CH₃)Ph, the C₁₋₂₀-alkyl radicals, preferably —CH₃, —CH₂CH₃, —CH₂CH₂CH₃, —CH(CH₃)₂, —CH₂CH₂CH₂H₃, —CH₂CH(CH₃)₂, —CH(CH₃)CH₂CH₃, —C(CH₃)₃, and A represents an anion which is preferably selected from chloride, bromide, iodide or methosulfate.

Particularly preferred hair-treatment compositions according to the invention are characterized in that they comprise at least one aminofunctional silicone of the formula (IIa) 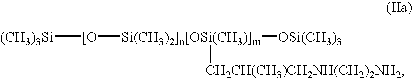
in which m and n are numbers whose sum (m+n) is between 1 and 2,000, preferably between 50 and 150, where n preferably assumes values of from 0 to 1,999 and in particular, from 49 to 149 and m preferably assumes values of from 1 to 2,000, in particular, from 1 to 10.

These silicones are referred to as trimethylsilylamodimethicones according to the INCI declaration.

Particular preference is also given to hair-treatment compositions according to the invention which comprise at least one aminofunctional silicone of the formula (IIb) 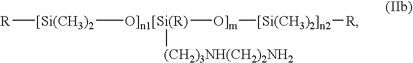
in which R is —OH, —O—CH₃ or a —CH₃ group, and m, n1 and n2 are numbers whose sum (m+n1+n2) is between 1 and 2,000, preferably between 50 and 150, where the sum (n1+n2) preferably assumes values from 0 to 1,999 and in particular, from 49 to 149 and m preferably assumes values of from 1 to 2,000, in particular, from 1 to 10.

These silicones are referred to as amodimethicones according to the INCI declaration.

Irrespective of which aminofunctional silicones are used, preference is given to hair-treatment compositions according to the invention which comprise an aminofunctional silicone whose amine number is above 0.25 meq/g, preferably above 0.3 meq/g and in particular, above 0.4 meq/g. The amine number here is the milli-equivalents of amine per gram of aminofunctional silicone. It can be determined by titration and also quoted in the unit mg KOH/g.

Hair-treatment compositions preferred according to the invention are ones which, based on their weight, comprise 0.01 to 10% by weight, preferably 0.1 to 8% by weight, particularly preferably 0.25 to 7.5% by weight and in particular, 0.5 to 5% by weight, of aminofunctional silicone(s).

Cyclic dimethicones referred to in accordance with INCI as CYCLOMETHICONE can also advantageously be used according to the invention. Here, preference is given to hair-treatment compositions according to the invention which comprise at least one silicone of the formula III 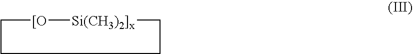
in which x is a number from 3 to 200, preferably from 3 to 10, further preferably from 7 and in particular, 4, 5 or 6.

The silicones described above have a backbone which is constructed from —Si—O—Si— units. These Si—O—Si units can of course also be interrupted by carbon chains. Corresponding molecules are accessible by chain-extension reactions and are preferably used in the form of silicone-in-water emulsions.

The silicone-in-water emulsions which can be used according to the invention can be produced by known methods, as are disclosed, for example, in U.S. Pat. No. 5,998,537 and EP 0 874 017 A1.

In summary, this production method involves the emulsifying mixing of components, one of which comprises at least one polysiloxane, the other of which comprises at least one organosilicone material which reacts with the polysiloxane in a chain-extension reaction, where at least one metal-ion-containing catalyst for the chain-extension reaction, at least one surfactant and water are present.

Chain-extension reactions with polysiloxanes are known and can involve, for example, the hydrosilylation reaction in which an Si—H group reacts with an aliphatically unsaturated group in the presence of a platinum/rhodium catalyst to form polysiloxanes with some Si—(C)ₚ—Si bonds (p=1-6), where the polysiloxanes are also referred to as polysiloxane-polysilalkylene copolymers.

The chain-extension reaction can also involve the reaction of an Si—OH group (for example, a hydroxy-terminated polysiloxane) with an alkoxy group (for example, alkoxysilanes, silicates or alkoxysiloxanes) in the presence of a metal-containing catalyst to form polysiloxanes.

The polysiloxanes which are used in the chain-extension reaction include a substantially linear polymer of the following structure:
R—Si(R₂)—[—O—Si(R₂)—]ₙ—O—SiR₃

In this structure, each R, independently of the others, is a hydrocarbon radical having up to 20 carbon atoms, preferably having 1 to 6 carbon atoms, such as, for example, an alkyl group (for example, methyl, ethyl, propyl or butyl), an aryl group (for example, phenyl), or the group required for the chain-extension reaction (“reactive group”, for example, Si-bonded H atoms, aliphatically unsaturated groups, such as vinyl, allyl or hexenyl, hydroxy, alkoxy, such as methoxy, ethoxy or propoxy, alkoxy-alkoxy, acetoxy, amino etc.), with the proviso, that on average, one to two reactive groups are present per polymer, n is a positive number>1. Preferably, a majority of the reactive groups, particularly preferably >90%, and in particular, >98%, of the reactive groups is bonded to the terminal Si atoms in the siloxane. Preferably, n is numbers which describe polysiloxanes which have viscosities between 1 and 1,000,000 mm²/s, particularly preferably viscosities between 1,000 and 100,000 mm²/s.

The polysiloxanes can be branched to a slight degree (for example, <2 mol % of the siloxane units), but the polymers are substantially linear, particularly preferably completely linear. Furthermore, the substituents R can in turn be substituted, for example, by N-containing groups (for example, amino groups), epoxy groups, S-containing groups, Si-containing groups, O-containing groups etc. Preferably, at least 80% of the radicals R are alkyl radicals, particularly preferably methyl groups.

The organosilicone material which reacts with the polysiloxane in the chain-extension reaction can either be a second polysiloxane or a molecule which acts as chain extender. If the organosilicone material is a polysiloxane, it has the general structure mentioned above. In these cases, a polysiloxane in the reaction has (at least) one reactive group, and a second polysiloxane has (at least) a second reactive group which reacts with the first group.

If the organosilicone material comprises a chain-extension agent, this may be one material, such as, for example, a silane, a siloxane (for example, disiloxanes or trisiloxane) or a silazane. Thus, for example, a composition which comprises a polysiloxane according to the general structure described above which has at least one Si—OH group can be chain-extended by reacting it with an alkoxysilane (for example, a dialkoxysilane or trialkoxysilane) in the presence of tin- or titanium-containing catalysts.

The metal-containing catalysts in the chain-extension reaction are mostly specific for a certain reaction. Such catalysts are known in the prior art and comprise, for example, metals, such as platinum, rhodium, tin, titanium, copper, lead, etc. In a preferred chain-extension reaction, a polysiloxane with at least one aliphatically unsaturated group, preferably an end group, is reacted with an organosilicone material in the presence of a hydrosilylation catalyst which is a siloxane or polysiloxane with at least one (preferably terminal) Si—H group. The polysiloxane has at least one aliphatically unsaturated group and satisfies the general formula given above in which R and n are as defined above, where, on average, between 1 and 2 groups R have one aliphatically unsaturated group per polymer. Representative aliphatically unsaturated groups are, for example, vinyl, allyl, hexenyl or cyclohexenyl or a group R²CH═CHR³, in which R² is a divalent aliphatic chain bonded to the silicon and R³ is a hydrogen atom or an alkyl group. The organosilicone material with at least one Si—H group preferably has the abovementioned structure in which R and n are as defined above and where, on average, between 1 and 2 groups R are a hydrogen and n is 0 or a positive integer.

This material can be a polymer or a low molecular weight material such as a siloxane (for example, a disiloxane or a trisiloxane).

The polysiloxane having at least one aliphatically unsaturated group and the organosilicone material having at least one Si—H group react in the presence of a hydrosilylation catalyst. Such catalysts are known from the prior art and include, for example, platinum- and rhodium-containing materials. The catalysts can assume any known form, for example, platinum or rhodium applied to support materials (such as, for example, silica gel or activated carbon), or other suitable compounds, such as platinum chloride, salts of platinic or chloroplatinic acids. A catalyst preferred on account of the good dispersibility in organosilicone systems and the slight color changes is chloroplatinic acid either in the form of the commercially available hexahydrate or in anhydrous form.

In a further preferred chain-extension reaction, a polysiloxane having at least one Si—OH group, preferably an end group, is reacted with an organosilicone material which has at least one alkoxy group, preferably a siloxane having at least one Si—OR group or an alkoxy silane having at least two alkoxy groups. Here, the catalyst used is again a metal-containing catalyst.

For the reaction of an Si—OH group with an Si—OR group there are many catalysts known in the literature, for example, organometallic compounds, such as organotin salts, titanates or titanium chelates and complexes. Examples include tin octoate, dibutyl tin dilaurate, dibutyltin diacetate, dimethyltin dineodecanoate, dibutyltin dimethoxide, isobutyltin triceroate, dimethyltin dibutyrate, dimethyltin dineodecanoate, triethyltin tartrate, tin oleate, tin naphthenate, tin butyrate, tin acetate, tin benzoate, tin sebacate, tin succinate, tetrabutyl titanate, tetraisopropyl titanate, tetraphenyl titanate, tetraoctadecyl titanate, titanium naphthanate, ethyltriethanolamine titanate, titanium diisopropyldiethylacetoacetate, titanium diisopropoxydiacetylacetonate and titanium tetraalkoxides in which the alkoxide is butoxy or propoxy.

The silicone-in-water emulsions moreover preferably comprise at least one surfactant. These have been described in detail above.

Hair-treatment compositions likewise preferred according to the invention are characterized in that they comprise at least one silicone of the formula IV
R₃Si—[O—SiR₂]ₓ—(CH₂)ₙ—[O—SiR₂]_{y}—O—SiR₃ (IV),
in which R is identical or different radicals from the group —H, -phenyl, -benzyl, —CH₂—CH(CH₃)Ph, the C₁₋₂₀-alkyl radicals, preferably —CH₃, —CH₂CH₃, —CH₂CH₂CH₃, —CH(CH₃)₂, —CH₂CH₂CH₂H₃, —CH₂CH(CH₃)₂, —CH(CH₃)CH₂CH₃, —C(CH₃)₃, x and y are a number from 0 to 200, preferably from 0 to 10, more preferably from 0 to 7 and in particular, 0, 1, 2, 3, 4, 5 or 6, and n is a number from 0 to 10, preferably from 1 to 8 and in particular, is 2, 3, 4, 5, 6.

The silicones are preferably water-soluble. Hair-treatment compositions preferred according to the invention are therefore characterized in that they can additionally comprise a water-soluble silicone.

In a further preferred embodiment, the compositions according to the invention can comprise emulsifiers (F). At the phase interface, emulsifiers bring about the formation of water- or oil-stable adsorption layers which protect the dispersed droplets against coalescence and thus stabilize the emulsion. Emulsifiers, like surfactants, are therefore constructed from a hydrophobic molecular moiety and a hydrophilic molecular moiety. Hydrophilic emulsifiers form preferably O/W emulsions and hydrophobic emulsifiers form preferably W/O emulsions. An emulsion is understood as meaning a droplet-like distribution (dispersion) of one liquid in another liquid with the expenditure of energy to produce stabilizing phase interfaces by means of surfactants. The choice of these emulsifying surfactants or emulsifiers is governed here by the substances to be dispersed and the particular outer phase, and also the finely divided nature of the emulsion. Emulsifiers which can be used according to the invention are, for example,
addition products of from 4 to 30 mol of ethylene oxide and/or 0 to 5 mol of propylene oxide onto linear fatty alcohols having 8 to 22 carbon atoms, onto fatty acids having 12 to 22 carbon atoms and onto alkylphenols having 8 to 15 carbon atoms in the alkyl group,
C₁₂-C₂₂-fatty acid mono- and diesters of addition products of from 1 to 30 mol of ethylene oxide onto polyols having 3 to 6 carbon atoms, in particular, onto glycerol,
ethylene oxide and polyglycerol addition products onto methyl glucoside fatty acid esters, fatty acid alkanolamides and fatty acid glucamides,
C₈-C₂₂-alkyl mono- and oligoglycosides and ethoxylated analogs thereof, where degrees of oligomerization of from 1.1 to 5, in particular, 1.2 to 2.0, and glucose as sugar component are preferred,
mixtures of alkyl (oligo)glucosides and fatty alcohols, for example, the commercially available product Montanov®68,
addition products of from 5 to 60 mol of ethylene oxide onto castor oil and hydrogenated castor oil,
partial esters of polyols having 3-6 carbon atoms with saturated fatty acids having 8 to 22 carbon atoms,
sterols. Sterols are understood as meaning a group of steroids which carry a hydroxyl group on carbon atom 3 of the steroid backbone and are isolated either from animal tissue (zoosterols) or from vegetable fats (phytosterols). Examples of zoosterols are cholesterol and lanosterol. Examples of suitable phytosterols are ergosterol, stigmasterol and sitosterol. Sterols are also isolated from fungi and yeasts, the so-called mycosterols.
phospholipids. This is understood in particular, as meaning the glucose phospholipids, which are obtained, for example, as lecithins or phosphatidylcholines from e.g., egg yolk or plant seeds (e.g., soya beans).
fatty acid esters of sugars and sugar alcohols, such as sorbitol,
polyglycerols and polyglycerol derivatives, such as, for example, polyglycerol poly-12-hydroxystearate (commercial product Dehymuls® PGPH),
linear and branched fatty acids having 8 to 30 carbon atoms and the Na, K, ammonium, Ca, Mg and Zn salts thereof.

The compositions according to the invention comprise the emulsifiers preferably in amounts of 0.1-25% by weight, in particular, 0.5-15% by weight, based on the total composition.

Preferably, the compositions according to the invention can comprise at least one nonionogenic emulsifier with an HLB value of from 8 to 18. Nonionogenic emulsifiers with an HLB value of 10-15 may be particularly preferred according to the invention.

It has also been shown to be advantageous if polymers (G) are present in the compositions according to the invention. In a preferred embodiment, polymers are therefore added to the compositions used according to the invention, with either cationic, anionic, amphoteric or nonionic polymers having proven to be effective.

Cationic and amphoteric polymers can preferably be used according to the invention. Cationic or amphoteric polymers are to be understood as meaning polymers which, in the main chain and/or side chain, have a group which may be “temporarily” or “permanently” cationic. According to the invention, the term “permanently cationic” is used to refer to those polymers which have a cationic group irrespective of the pH of the composition. These are generally polymers which contain a quaternary nitrogen atom, for example, in the form of an ammonium group. Preferred cationic groups are quaternary ammonium groups. In particular, those polymers in which the quaternary ammonium group is bonded via a C1-4 hydrocarbon group to a polymer main chain constructed from acrylic acid, methacrylic acid or derivatives thereof have proven to be particularly suitable.

Homopolymers of the general formula (G1-I) 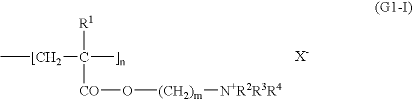
in which R¹=—H or —CH₃, R², R³ and R⁴, independently of one another, are chosen from C1-4-alkyl, -alkenyl or -hydroxyalkyl groups, m=1, 2, 3 or 4, n is a natural number and X⁻ is a physiologically compatible organic or inorganic anion, and copolymers consisting essentially of the monomer units listed in formula (G1-I), and nonionogenic monomer units, are particularly preferred cationic polymers. Among these polymers, preference is given according to the invention to those for which at least one of the following conditions applies:
R¹ is a methyl group
R², R³ and R⁴ are methyl groups
m has the value 2.

Suitable physiologically compatible counterions X⁻ are, for example, halide ions, sulfate ions, phosphate ions, methosulfate ions, and organic ions, such as lactate, citrate, tartrate and acetate ions. Preference is given to halide ions, in particular, chloride.

A particularly suitable homopolymer is, if desired crosslinked, poly(methacryloyloxyethyltrimethylammonium chloride) with the INCI name Polyquaternium-37. Such products are commercially available, for example, under the names Rheocare® CTH (Cosmetic Rheologies) and Synthalen® CR (Ethnichem). The crosslinking can take place if desired with the help of polyolefinically unsaturated compounds, for example, divinylbenzene, tetraallyloxyethane, methylenebisacrylamide, diallyl ether, polyallyl poly-glyceryl ether, or allyl ethers of sugars or sugar derivatives, such as erythritol, pentaerythritol, arabitol, mannitol, sorbitol, sucrose or glucose. Methylenebisacrylamide is a preferred crosslinking composition.

The homopolymer is preferably used in the form of a nonaqueous polymer dispersion which should have a polymer fraction not below 30% by weight. Such polymer dispersions are commercially available under the names Salcare® SC 95 (about 50% polymer fraction, further components: mineral oil (INCI name: Mineral Oil) and tridecyl polyoxypropylene polyoxyethylene ether (INCI name: PPG-1-Trideceth-6)) and Salcare® SC 96 (about 50% polymer fraction, further components: mixture of diesters of propylene glycol with a mixture of caprylic acid and capric acid (INCI name: Propylene Glycol Dicaprylate/Dicaprate) and tridecyl polyoxypropylene polyoxyethylene ether (INCI name: PPG-1-Trideceth-6)).

Copolymers with monomer units according to formula (G1-l) comprise, as nonionogenic monomer units, preferably acrylamide, methacrylamide, C₁₋₄-alkyl acrylates and C₁₋₄-alkyl methacrylates. Among these nonionogenic monomers, particular preference is given to acrylamide. As in the case of the homopolymers described above, these copolymers too may be crosslinked. A copolymer preferred according to the invention is the crosslinked acrylamide-methacryloyloxethyltrimethylammonium chloride copolymer. Such copolymers in which the monomers are present in a weight ratio of about 20:80 are commercially available as about 50% strength nonaqueous polymer dispersion under the name Salcare® SC 92.

Further preferred cationic polymers are, for example,
quaternized cellulose derivatives, as are commercially available under the names Celquat® and Polymer JR®. The compounds Celquat® H 100, Celquat® L 200 and Polymer JR®400 are preferred quaternized cellulose derivatives,
cationic alkyl polyglycosides as in DE-C 44 13 686,
cationized honey, for example, the commercial product Honeyquat® 50,
cationic guar derivatives, such as, in particular, the products sold under the trade names Cosmedia® Guar and Jaguar®,
polymeric dimethyldiallylammonium salts and copolymers thereof with esters and amides of acrylic acid and methacrylic acid. The products commercially available under the names Merquat® 100 (poly(dimethyldiallylammonium chloride)) and Merquat® 550 (dimethyldiallylammonium chloride-acrylamide copolymer) are examples of such cationic polymers,
copolymers of vinylpyrrolidone with quaternized derivatives of dialkylaminoalkyl acrylate and methacrylate, such as, for example, vinylpyrrolidone-dimethylaminoethyl methacrylate copolymers quaternized with diethyl sulfate. Such compounds are commercially available under the names Gafquat® 734 and Gafquat® 755,
vinylpyrrolidone-vinylimidazolium methochloride copolymers, as are supplied under the names Luviquat® FC 370, FC 550, FC 905 and HM 552,
quaternized polyvinyl alcohol,
and the polymers known under the names Polyquaternium 2, Polyquaternium 17, Polyquaternium 18 and Polyquaternium 27 with quaternary nitrogen atoms in the polymer main chain.

As cationic polymers it is likewise possible to use the polymers known under the names Polyquaternium-24 (commercial product e.g., Quatrisoft® LM 200). According to the invention, it is likewise possible to use the copolymers of vinylpyrrolidone, as are obtainable as commercial products Copolymer 845 (manufacturer: ISP), Gaffix® VC 713 (manufacturer: ISP), Gafquat® ASCP 1011, Gafquat® HS 110, Luviquat® 8155 and Luviquat® MS 370.

Further cationic polymers which can be used in the compositions according to the invention are the so-called “temporarily cationic” polymers. These polymers usually comprise an amino group which is present as quaternary ammonium group and thus in cationic form at certain pH values. Preference is given, for example, to chitosan and derivatives thereof, as are freely available commercially, for example, under the trade names Hydagen® CMF, Hydagen® HCMF, Kytamer® PC and Chitolam® NB/101.

Cationic polymers preferred according to the invention are cationic cellulose derivatives and chitosan and derivatives thereof, in particular, the commercial products Polymer® JR 400, Hydagen® HCMF and Kytamer® PC, cationic guar derivatives, cationic honey derivatives, in particular, the commercial product Honeyquat® 50, cationic alkyl polyglycosides as in DE-C 44 13 686 and polymers of the Polyquaternium-37 type.

In addition, cationized protein hydrolyzates are types of cationic polymers, where the parent protein hydrolyzate can originate from animal, for example, from collagen, milk or keratin, from plant, for example, from wheat, corn, rice, potatoes, soya or almonds, from marine life forms, for example, from fish collagen or algae, or protein hydrolyzates obtained by biotechnological methods. The protein hydrolyzates on which the cationic derivatives according to the invention are based can be obtained from the corresponding proteins by a chemical, in particular, alkaline or acidic, hydrolysis, by an enzymatic hydrolysis and/or a combination of both types of hydrolysis. The hydrolysis of proteins generally gives a protein hydrolyzate with a molecular weight distribution from about 100 daltons to several thousand daltons. Preference is given here to those cationic protein hydrolyzates whose parent protein moiety has a molecular weight of from 100 to 25,000 daltons, preferably 250 to 5,000 daltons. In addition, cationic protein hydrolyzates are to be understood as meaning quaternized amino acids and mixtures thereof. The quaternization of the protein hydrolyzates or of the amino acids is often carried out using quaternary ammonium salts, such as, for example, N,N-dimethyl-N-(n-alkyl)-N-(2-hydroxy-3-chloro-n-propyl)ammonium halides. In addition, the cationic protein hydrolyzates can also be yet further derivatized. Typical examples of the cationic protein hydrolyzates and derivatives according to the invention which may be mentioned are the products specified under the INCI names in the “International Cosmetic Ingredient Dictionary and Handbook”, (seventh edition 1997, The Cosmetic, Toiletry, and Fragrance Association 1101 17th Street, N.W., Suite 300, Washington, D.C. 20036-4702) and commercially available products: Cocodimonium Hydroxypropyl Hydrolyzed Collagen, Cocodimonium Hydroxypropyl Hydrolyzed Casein, Cocodimonium Hydroxypropyl Hydrolyzed Collagen, Cocodimonium Hydroxypropyl Hydrolyzed Hair Keratin, Cocodimonium Hydroxypropyl Hydrolyzed Keratin, Cocodimonium Hydroxypropyl Hydrolyzed Rice Protein, Cocodimonium Hydroxypropyl Hydrolyzed Soy Protein, Cocodimonium Hydroxypropyl Hydrolyzed Wheat Protein, Hydroxypropyl Arginine Lauryl/Myristyl Ether HCl, Hydroxypropyltrimonium Gelatin, Hydroxypropyltrimonium Hydrolyzed Casein, Hydroxypropyltrimonium Hydrolyzed Collagen, Hydroxypropyltrimonium Hydrolyzed Conchiolin Protein, Hydroxypropyltrimonium Hydrolyzed Keratin, Hydroxypropyltrimonium Hydrolyzed Rice Bran Protein, Hydroxypropyltrimonium Hydrolyzed Soy Protein, Hydroxypropyl Hydrolyzed Vegetable Protein, Hydroxypropyltimonium Hydrolyzed Wheat Protein, Hydroxypropyltrimonium Hydrolyzed Wheat Protein/Siloxysilicate, Hydroxypropyl Hydrolyzed Soy Protein, Laurdimonium Hydroxypropyl Hydrolyzed Wheat Protein, Laurdimonium Hydroxypropyl Hydrolyzed Wheat Protein/Siloxysilicate, Lauryldimonium Hydroxypropyl Hydrolyzed Casein, Lauryldimonium Hydroxypropyl Hydrolyzed Collagen, Lauryldimonium Hydroxypropyl Hydrolyzed Keratin, Lauryldimonium Hydroxypropyl Hydrolyzed Soy Protein, Steardimonium Hydroxypropyl Hydrolyzed Casein, Steardimonium Hydroxypropyl Hydrolyzed Collagen, Steardimonium Hydroxypropyl Hydrolyzed Keratin, Steardimonium Hydroxypropyl Hydrolyzed Rice Protein, Steardimonium Hydroxypropyl Hydrolyzed Soy Protein, Steardimonium Hydroxypropyl Hydrolyzed Vegetable Protein, Steardimonium Hydroxypropyl Hydrolyzed Wheat Protein, Steartrimonium Hydroxyethyl Hydrolyzed Collagen, Quaternium-76 Hydrolyzed Collagen, Quaternium-79 Hydrolyzed Collagen, Quaternium-79 Hydrolyzed Keratin, Quaternium-79 Hydrolyzed Milk Protein, Quaternium-79 Hydrolyzed Soy Protein, Quaternium-79 Hydrolyzed Wheat Protein.

Very particular preference is given to the plant-based cationic protein hydrolyzates and derivatives.

In addition to cationic polymers, or instead of them, the compositions according to the invention can also comprise amphoteric polymers. These additionally have at least one negatively charged group in the molecule and are also referred to as zwitterionic polymers. Zwitterionic polymers which can preferably be used within the scope of the present invention are essentially composed of

A) monomers with quaternary ammonium groups of the general formula (Z-I),
R¹—CH═CR²—CO-Z-(CₙH₂ₙ)—N⁽⁺⁾R³R⁴R⁵A⁽⁻⁾ (Z-I)
in which R¹ and R², independently of one another, are hydrogen or a methyl group and R³, R⁴ and R⁵, independently of one another, are alkyl groups having 1 to 4 carbon atoms, Z is an NH group or an oxygen atom, n is an integer from 2 to 5 and A⁽⁻⁾ is the anion of an organic or inorganic acid
and
   B) monomeric carboxylic acids of the general formula (Z-II),
   R⁶—CH═CR⁷—COOH (II)
in which R⁶ and R⁷, independently of one another, are hydrogen or methyl groups.

Suitable starting monomes are, for example, dimethylaminoethylacrylamide, dimethylaminoethylmethacrylamide, dimethylaminopropylacrylamide, dimethylaminopropylmethacrylamide and diethylaminoethylacrylamide if Z is an NH group, or dimethylaminoethyl acrylate, dimethylaminoethyl methacrylate and diethylaminoethyl acrylate if Z is an oxygen atom.

The monomers containing a tertiary amino group are then quaternized in a known manner, where methyl chloride, dimethyl sulfate or diethyl sulfate are particularly suitable as alkylating reagents. The quaternization reaction can take place in aqueous solution or in the solvent.

Advantageously, monomers of the formula (Z-I) which are derivatives of acrylamide or methacrylamide are used. Preference is also given to those monomers which comprise, as counterions, halide, methoxysulfate or ethoxysulfate ions. Preference is likewise given to those monomers of the formula (Z-I) in which R³, R⁴ and R⁵ are methyl groups.

The acrylamidopropyltrimethylammonium chloride is a very particularly preferred monomer of the formula (Z-I).

Suitable monomeric carboxylic acids of the formula (Z-II) are acrylic acid, methacrylic acid, crotonic acid and 2-methylcrotonic acid. Preference is given to using acrylic or methacrylic acid, in particular, acrylic acid.

The zwitterionic polymers which can be used according to the invention are prepared from monomers of the formulas (Z-I) and (Z-II) by polymerization methods known per se. The polymerization can take place either in aqueous or aqueous-alcoholic solution. The alcohols used are alcohols having 1 to 4 carbon atoms, preferably isopropanol, which simultaneously serve as polymerization regulators. However, other components can also be added to the monomer solution as regulator, e.g., formic acid or mercaptans, such as thioethanol and thioglycolic acid. The polymerization is initiated with the help of radical-forming substances. For this purpose, it is possible to use redox systems and/or thermally decomposing radical formers of the azo compound type, such as, for example, azoisobutyronitrile, azobis(cyanopentanoic acid) or azobis(amidinopropane) dihydrochloride. Suitable redox systems are, for example, combinations of hydrogen peroxide, potassium or ammonium peroxodisulfate, and tertiary butyl hydroperoxide with sodium sulfite, sodium dithionite or hydroxylamine hydrochloride as reduction component.

The polymeization can be carried out isothermally or under adiabatic conditions, where, depending on the concentration ratios, the temperature range for the course of the reaction can vary between 20 and 200° C. as a result of the heat of polymerization which is liberated, and the reaction, if appropriate, has to be carried out under the superatmospheric pressure which is established. Preferably, the reaction temperature is between 20 and 100° C.

The pH during the copolymerization can vary within a wide range. Advantageously, polymerization is carried out at a low pH; however, a pH above neutral is also possible. After the polymerization, an aqueous base, e.g., sodium hydroxide solution, potassium hydroxide solution or ammonia, is used to adjust the pH to between 5 and 10, preferably 6 to 8. Further details relating to the polymerization method can be found in the examples.

Polymers which have proven particularly effective are those in which the monomers of the formula (Z-I) were present in excess compared to the monomers of the formula (Z-II). It is therefore preferred according to the invention to use those polymers which consist of monomers of the formula (Z-I) and the monomers of the formula (Z-II) in a molar ratio of from 60:40 to 95:5, in particular, from 75:25 to 95:5.

The cationic and amphoteric polymers are present in the compositions according to the invention preferably in amounts of from 0.05 to 10% by weight, based on the total composition. Amounts of from 0.1 to 5% by weight are particularly preferred.

The anionic polymers (G2) are anionic polymers which have carboxylate and/or sulfonate groups. Examples of anionic monomers of which such polymers can consist are acrylic acid, methacrylic acid, crotonic acid, maleic anhydride and 2-acrylamido-2-methylpropanesulfonic acid. In this connection, the acid groups can be completely or partly in the form of the sodium, potassium, ammonium, mono- or triethanolammonium salt. Preferred monomers are 2-acrylamido-2-methylpropanesulfonic acid and acrylic acid.

Anionic polymers which have proven very particularly effective are those which comprise 2-acrylamido-2-methylpropanesulfonic acid as the sole monomer or comonomer, where the sulfonic acid group may be present completely or partly in the form of the sodium, potassium, ammonium, mono- or triethanolammonium salt.

Particular preference is given to the homopolymer of 2-acrylamido-2-methyl-propanesulfonic acid, which is commercially available, for example, under the name Rheothik® 11-80.

Within this embodiment, it may be preferred to use copolymers of at least one anionic monomer and at least one nonionogenic monomer. With regard to the anionic monomers, reference is made to the substances listed above. Preferred nonionogenic monomers are acrylamide, methacrylamide, acrylic esters, methacrylic esters, vinylpyrrolidone, vinyl ethers and vinyl esters.

Preferred anionic copolymers are acrylic acid-acrylamide copolymers and in particular, polyacrylamide copolymers with monomers containing sulfonic acid groups. A particularly preferred anionic copolymer consists of 70 to 55 mol % of acrylamide and 30 to 45 mol % of 2-acrylamido-2-methylpropanesulfonic acid, where the sulfonic acid group is present completely or partly in the form of the sodium, potassium, ammonium, mono- or triethanolammonium salt. This copolymer can also be in crosslinked form, in which case suitable crosslinking compositions are preferably polyolefinically unsaturated compounds such as tetraallyloxyethane, allyl sucrose, allyl pentaerythritol and methylenebisacrylamide. Such a polymer is present in the commercial product Sepigel®305 from SEPPIC. The use of this compound which, besides the polymer component, comprises a hydrocarbon mixture (C₁₃-C₁₄-isoparaffin) and a nonionogenic emulsifier (Laureth-7) has proven particularly advantageous within the scope of the teaching according to the invention.

The sodium acryloyldimethyltaurate copolymers sold under the name Simulgel®600 as compound with isohexadecane and polysorbate-80 have also proven to be particularly effective according to the invention.

Likewise preferred anionic homopolymers are uncrosslinked and crosslinked polyacrylic acids. Here, allyl ethers of pentaerythritol, of sucrose and of propylene may be preferred crosslinking compositions. Such compounds are commercially available, for example, under the trade name Carbopol®.

Copolymers of maleic anhydride and methyl vinyl ether, in particular, those with crosslinks, are likewise color-retaining polymers. A maleic acid-methyl vinyl ether copolymer crosslinked with 1,9-decadienes is commercially available under the name Stabileze® QM.

In addition, polymers which can be used for increasing the effect of the active ingredient complex (A) according to the invention are amphoteric polymers (G3). The term amphoteric polymers includes both those polymers which comprise both free amino groups and also free —COOH or SO₃H groups in the molecule and are capable of forming internal salts, and also zwitterionic polymers which comprise quaternary ammonium groups and —COO⁻ or —SO₃⁻ groups in the molecule, and those polymers which comprise —COOH or SO₃H groups and quaternary ammonium groups.

One example of an amphopolymer which can be used according to the invention is the acrylic resin obtainable under the name Amphomer®, which is a copolymer of tert-butylaminoethyl methacrylate, N-(1,1,3,3-tetramethylbutyl)acrylamide and two or more monomers from the group consisting of acrylic acid, methacrylic acid and monoesters thereof.

Preferably used amphoteric polymers are those polymers which consist essentially of

(a) monomers with quaternary ammonium groups of the general formula (G3-I),
R¹—CH═CR²—CO-Z-(CₙH₂ₙ)—N⁽⁺⁾R³R⁴R⁵A⁽⁻⁾ (G3-I)
in which R¹ and R², independently of one another, are hydrogen or a methyl group and R³, R⁴ and R⁵, independently of one another, are alkyl groups having 1 to 4 carbon atoms, Z is an NH group or an oxygen atom, n is an integer from 2 to 5 and A⁽⁻⁾ is the anion of an organic or inorganic acid, and
(b) monomeric carboxylic acids of the general formula (G3-II),
R⁶—CH═CR⁷—COOH (G3-II)
in which R⁶ and R⁷, independently of one another, are hydrogen or methyl groups.

According to the invention, these compounds can either be used directly or in salt form, which is obtained by neutralization of the polymers, for example, with an alkali metal hydroxide. Very particular preference is given to using those polymers in which monomers of type (a) are used, in which R³, R⁴ and R⁵ are methyl groups, Z is an NH group and A⁽⁻⁾ is a halide, methoxysulfate or ethoxysulfate ion; acrylamidopropyltrimethyl-ammonium chloride is a particularly preferred monomer (a). The monomer (b) used for the specific polymers is preferably acrylic acid.

In a further embodiment, the compositions according to the invention can comprise nonionogenic polymers (G4).

Suitable nonionogenic polymers are, for example:
vinylpyrrolidone/vinyl ester copolymers, as are sold, for example, under the trade name Luviskol® (BASF). Luviskol® VA 64 and Luviskol® VA 73, in each case vinylpyrrolidone/vinyl acetate copolymers, are likewise preferred nonionic polymers.
cellulose ethers, such as hydroxypropylcellulose, hydroxyethylcellulose and methyl-hydroxypropylcellulose, as are sold, for example, under the trade name Culminal® and Benecel® (AQUALON) and Natrosol® grades (Hercules)
starch and derivatives thereof, in particular, starch ethers, for example, Structure® XL (National Starch), a multifunctional, salt-tolerant starch;
shellac
polyvinylpyrrolidones, as are sold, for example, under the name Luviskol® (BASF).
siloxanes. These siloxanes may either be water-soluble or water-insoluble. Of suitability are both volatile and nonvolatile siloxanes, where nonvolatile siloxanes are understood as meaning those compounds whose boiling point at atmospheric pressure is above 200° C. Preferred siloxanes are polydialkylsiloxanes, such as, for example, polydimethylsiloxane, polyalkylarylsiloxanes, such as, for example, polyphenyl-methylsiloxane, ethoxylated polydialkylsiloxanes, and polydialkylsiloxanes which contain amine and/or hydroxy groups.
glycosidically substituted silicones.

According to the invention, it is also possible for the preparations used to comprise a plurality of, in particular, two, different polymers of identical charge and/or in each case one ionic and one amphoteric and/or nonionic polymer.

The polymers (G) are present in the compositions according to the invention preferably in amounts of from 0.05 to 10% by weight, based on the total composition. Amounts of from 0.1 to 5% by weight, in particular, from 0.1 to 3% by weight, are particularly preferred.

In addition to the specified substances, the compositions according to the invention can comprise further care substances. These are particularly advantageously, for example, vitamins, provitamins or vitamin precursors, meaning that compositions preferred according to the invention are characterized in that they additionally comprise at least one substance from the group of vitamins, provitamins and vitamin precursors, and derivatives thereof, preference being given to vitamins, provitamins and vitamin precursors which are assigned to the groups A, B, C, E, F and H. These have been described in detail above.

A further group of care substances which may be present in the compositions according to the invention are the protein hydrolyzates and derivatives thereof (P). Protein hydrolyzates are product mixtures which are obtained by acidically, basically or enzymatically catalyzed degradation of proteins. According to the invention, the term protein hydrolyzates is also understood as meaning total hydrolyzates, and individual amino acids and derivatives thereof, and mixtures of different amino acids. Furthermore, according to the invention, polymers constructed from amino acids and amino acid derivatives are covered by the term protein hydrolyzates. The latter include, for example, polyalanine, polyasparagine, polyserine etc. Further examples of compounds which can be used according to the invention are L-alanyl-L-proline, polyglycine, glycyl-L-glutamine or D/L-methionine-5-methyl-sulfonium chloride. According to the invention, it is of course also possible to use β-aminoacids and derivatives thereof, such as β-alanine, anthranilic acid or hippuric acid. The molecular weight of the protein hydrolyzates which can be used according to the invention is between 75, the molecular weight of glycine, and 200,000; preferably, the molecular weight is 75 to 50,000 and very particularly preferably 75 to 20,000 daltons.

According to the invention, protein hydrolyzates both of vegetable and animal or marine or synthetic origin may be used.

Animal protein hydrolyzates are, for example, elastin, collagen, keratin and milk protein hydrolyzates, which may also be in the form of salts. Such products are sold, for example, under the trade names Dehylan® (Cognis), Promois® (Interorgana), Collapuron® (Cognis), Nutrilan® (Cognis), Gelita-Sol® (Deutsche Gelatine Fabriken Stoess & Co), Lexein® (Inolex) and Kerasol® (Croda).

According to the invention, preference is given to the use of protein hydrolyzates of vegetable origin, e.g., soya, almond, pea, potato and wheat protein hydrolyzates. Such products are available, for example, under the trade names Gluadin® (Cognis), DiaMin® (Diamalt), Lexein® (Inolex), Hydrosoy® (Croda), Hydrolupin® (Croda), Hydrosesame® (Croda), Hydrotritium® (Croda) and Crotein® (Croda).

Although the use of the protein hydrolyzates as such is preferred, it is also possible, instead of them, if appropriate to use amino acid mixtures obtained in another way. The use of derivatives of the protein hydrolyzates, for example, in the form of their fatty acid condensation products, is likewise possible. Such products are sold, for example, under the names Lamepon® (Cognis), Lexein® (Inolex), Crolastin® (Croda) or Crotein® (Croda).

The teaching according to the invention of course includes all isomeric forms, such as cis-, trans-isomers, diastereomers and chiral isomers.

According to the invention, it is also possible to use a mixture of two or more protein hydrolyzates (P).

The protein hydrolyzates (P) are present in the compositions in concentrations of from 0.01% by weight to 20% by weight, preferably from 0.05% by weight to 15% by weight and very particularly preferably in amounts of from 0.05% by weight to 5% by weight.

Furthermore, in a preferred embodiment of the invention, a composition according to the invention can also comprise UV filters (I). The UV filters to be used according to the invention are not subject to any general limitations with regard to their structure and their physical properties. Rather, all UV filters which can be used in the cosmetics sector and whose absorption maximum is in the UVA (315-400 nm) region, in the UVB (280-315 nm) region or in the UVC (<280 nm) region are suitable. UV filters with an absorption maximum in the UVB region, in particular, in the range from about 280 to about 300 nm, are particularly preferred.

The UV filters used according to the invention can be chosen, for example, from substituted benzophenones, p-aminobenzoic acid esters, diphenylacrylic acid esters, cinnamic acid esters, salicylic acid esters, benzimidazoles and o-aminobenzoic acid esters.

Examples of UV filters which can be used according to the invention are 4-aminobenzoic acid, N,N,N-trimethyl-4-(2-oxoborn-3-ylidenemethyl)aniline methylsulfate, 3,3,5-trimethylcyclohexyl salicylate (homosalate), 2-hydroxy-4-methoxybenzophenone (benzophenone-3; Uvinul®M 40, Uvasorb®MET, Neo Heliopan®BB, Eusolex®4360), 2-phenylbenzimidazole-5-sulfonic acid and the potassium, sodium and triethanolamine salts thereof (phenylbenzimidazolesulfonic acid; Parsol®HS; Neo Heliopan®Hydro), 3,3′-(1,4-phenylenedimethylene)bis(7,7-dimethyl-2-oxobicyclo[2.2.1]hept-1-ylmethanesulfonic acid) and salts thereof, 1-(4-tert-butylphenyl)-3-(4-methoxyphenyl)propane-1,3-dione (butyl methoxydibenzoylmethane; Parsol®1789, Eusolex®9020), α-(2-oxoborn-3-ylidene)toluene-4-sulfonic acid and salts thereof, ethoxylated ethyl 4-aminobenzoate (PEG-25 PABA; Uvinul®P 25), 2-ethylhexyl 4-dimethylaminobenzoate (octyl dimethyl PABA; Uvasorb®DMO, Escalol®507, Eusolex®6007), 2-ethylhexyl salicylate (octyl salicylate; Escalol®587, Neo Heliopan®OS, Uvinul®018), isopentyl 4-methoxycinnamate (isoamyl p-methoxycinnamate; Neo Heliopan®E 1000), 2-ethylhexyl 4-methoxycinnamate (octyl methoxycinnamate; Parsol®MCX, Escalol®557, Neo Heliopan®AV), 2-hydroxy-4-methoxybenzophenone-5-sulfonic acid and sodium salt thereof (benzophenone-4; Uvinol®MS 40; Uvasorb®S 5), 3-(4′-methylbenzylidene)-D,L-camphor (4-methylbenzylidene camphor; Parsol®5000, Eusolex®6300), 3-benzylidenecamphor, 4-isopropylbenzyl salicylate, 2,4,6-trianilino(p-carbo-2′-ethylhexyl-1′-oxy)-1,3,5-triazine, 3-imidazol-4-ylacrylic acid and the ethyl ester thereof, polymers of N-{(2 and 4)-[2-oxoborn-3-ylidenemethyl)benzyl}acrylamide, 2,4-dihydroxy-benzophenone (benzophenone-1; Uvasorb®20H, Uvinol®400), 1,1′-diphenylacrylonitrile acid 2-ethylhexyl ester (octocrylene; Eusolex®OCR, Neo Heliopan®Type 303, Univul®N 539 SG), menthyl o-aminobenzoate (menthyl anthranilate; Neo Heliopan®MA), 2,2′,4,4′-tetrahydroxybenzophenone (benzophenone-2; Uvinul®D-50), 2,2′-dihydroxy-4,4′-dimethoxybenzophenone (benzophenone-6), 2,2′-dihydroxy-4,4′-dimethoxybenzophenone-5 sodium sulfonate and 2′-ethylhexyl 2-cyano-3,3-diphenylacrylate. Preference is given to 4-aminobenzoic acid, N,N,N-trimethyl-4-(2-oxoborn-3-ylidenemethyl)aniline methylsulfate, 3,3,5-trimethylcyclohexyl salicylate, 2-hydroxy-4-methoxybenzophenone, 2-phenylbenzimidazole-5-sulfonic acid and the potassium, sodium and triethanolamine salts thereof, 3,3′-(1,4-phenylenedimethylene)bis(7,7-dimethyl-2-oxobicyclo[2.2.1]hept-1-ylmethanesulfonic acid) and salts thereof, 1-(4-tert-butylphenyl)-3-(4-methoxyphenyl)propane-1,3-dione, α-(2-oxoborn-3-ylidene)toluene-4-sulfonic acid and salts thereof, ethoxylated ethyl 4-aminobenzoate, 2-ethyl hexyl 4-dimethylaminobenzoate, 2-ethylhexyl salicylate, isopentyl 4-methoxycinnamate, 2-ethylhexyl 4-methoxycinnamate, 2-hydroxy-4-methoxybenzophenone-5-sulfonic acid and the sodium salt thereof, 3-(4′-methylbenzylidene)-D,L-camphor, 3-benzylidenecamphor, 4-isopropylbenzylsalicylate, 2,4,6-trianilino(p-carbo-2′-ethylhexyl-1′-oxy)-1,3,5-triazine, 3-imidazol-4-ylacrylic acid and the ethyl ester thereof, polymers of N-{(2 and 4)-[2-oxoborn-3-ylidenemethyl]benzyl}acrylamide. According to the invention, very particular preference is given to 2-hydroxy-4-methoxybenzophenone, 2-phenylbenzimidazole-5-sulfonic acid and the potassium, sodium and triethanolamine salts thereof, 1-(4-tert-butylphenyl)-3-(4-methoxyphenyl)propane-1,3-dione, 2-ethylhexyl 4-methoxycinnamate and 3-(4′-methylbenzylidene)-D,L-camphor.

Preference is given to those UV filters whose molar extinction coefficient is at the absorption maximum above 15,000, in particular, above 20,000.

Furthermore, it has been found that for structurally similar UV filters, in many cases the water-insoluble compound within the scope of the teaching according to the invention has the higher effect compared to those water-soluble compounds which differ from it by virtue of one or more additional ionic groups. For the purposes of the invention, water-insoluble UV filters are to be understood as meaning those which dissolve in water at 20° C. to not more than 1% by weight, in particular, to not more than 0.1% by weight. Furthermore, these compounds should be soluble in customary cosmetic oil components at room temperature to at least 0.1% by weight, in particular, to at least 1% by weight. The use of water-insoluble UV filters can therefore be preferred according to the invention.

According to a further embodiment of the invention, preference is given to those UV filters which have a cationic group, in particular, a quaternary ammonium group.

These UV filters have the general structure U-Q.

The structural moiety U is here a group which absorbs UV rays. This group can in principle be derived from the known abovementioned UV filters which can be used in the cosmetics sector by replacing one group, generally a hydrogen atom, of the UV filter with a cationic group Q, in particular, with a quaternary amino function.

Compounds from which the structural moiety U can be derived are, for example,

substituted benzophenones,

p-aminobenzoic acid esters,

diphenylacrylic acid esters,

cinnamic acid esters,

salicylic acid esters,

benzimidazoles and

o-aminobenzoic acid esters.

Structural moieties U which are derived from cinnamide or from N,N-dimethylaminobenzoamide are preferred according to the invention.

The structural moieties U can in principle be chosen so that the absorption maximum of the UV filters can be both in the UVA (315-400 nm) region, or in the UVB (280-315 nm) region or in the UVC (<280 nm) region. UV filters with an absorption maximum in the UVB region, in particular, in the range from about 280 to about 300 nm, are particularly preferred.

In addition, the structural moiety U is chosen, also depending on structural moiety Q, preferably such that the molar extinction coefficient of the UV filter at the absorption maximum is above 15,000, in particular, above 20,000.

The structural moiety Q comprises, as cationic group, preferably a quaternary ammonium group. This quaternary ammonium group can in principle be joined directly to the structural moiety U, meaning that the structural moiety U is one of the four substituents of the positively charged nitrogen atom. However, one of the four substituents on the positively charged nitrogen atom is preferably a group, in particular, an alkylene group having 2 to 6 carbon atoms, which functions as linkage between the structural moiety U and the positively charged nitrogen atom.

Advantageously, the group Q has the general structure —(CH₂)ₓ—N⁺R¹R²R³X⁻, in which x is an integer from 1 to 4, R¹ and R², independently of one another, are C₁₋₄-alkyl groups, R³ is a C₁₋₂₂-alkyl group or a benzyl group and X⁻ is a physiologically compatible anion. Within the context of this general structure, x is preferably 3, R¹ and R² are in each case a methyl group and R³ is either a methyl group or a saturated or unsaturated, linear or branched hydrocarbon chain having 8 to 22, in particular, 10 to 18, carbon atoms.

Physiologically compatible anions are, for example, inorganic anions, such as halides, in particular, chloride, bromide and fluoride, sulfate ions and phosphate ions, and organic anions, such as lactate, citrate, acetate, tartrate, methosulfate and tosylate.

Two preferred UV filters with cationic groups are the commercially available compounds cinnamic acid amidopropyltrimethylammonium chloride (Incroquat®UV-283) and dodecyldimethylaminobenzamidopropyldimethylammonium tosylate (Escalol®HP 610).

The teaching according to the invention of course also includes the use of a combination of two or more UV filters. Within the scope of this embodiment, the combination of at least one water-insoluble UV filter with at least one UV filter with a cationic group is preferred.

The UV filters (I) are present in the compositions used according to the invention usually in amounts of 0.1-5% by weight, based on the total composition. Amounts of 0.4-2.5% by weight are preferred.

The compositions according to the invention can also comprise a 2-pyrrolidinone-5-carboxylic acid and derivatives thereof (J). Preference is given to the sodium, potassium, calcium, magnesium or ammonium salts in which the ammonium ion carries one to three C₁- to C₄-alkyl groups besides hydrogen. The sodium salt is very particularly preferred. The amounts used in the compositions according to the invention are preferably 0.05 to 10% by weight, based on the total composition, particularly preferably 0.1 to 5% by weight, and in particular, 0.1 to 3% by weight.

Finally, the compositions according to the invention can also comprise plant extracts (L).

These extracts are usually prepared by extracting the whole plant. However, in individual cases, it may also be preferred to prepare the extracts exclusively from flowers and/or leaves of the plant.

With regard to the plant extracts which can be used according to the invention, reference is made in particular, to the extracts which are listed in the table starting on page 44 of the 3rd edition of the introduction to the ingredients declaration of cosmetic compositions, published by the Industrieverband Körperpflege-und Waschmittel e.V. (IKW), Frankfurt.

According to the invention, the extracts from green tea, oak bark, stinging nettle, hamamelis, hops, henna, camomile, burdock, horsetail, hawthorn, linden blossom, almond, aloe vera, spruce needle, horse chestnut, sandalwood, juniper, coconut, mango, apricot, lemon, wheat, kiwi, melon, orange, grapefruit, sage, rosemary, birch, mallow, lady's smock, wild thyme, yarrow, thyme, melissa, restharrow, coltsfoot, marshmallow, meristem, ginseng and ginger root in particular, are preferred.

Particular preference is given to the extracts from green tea, oak bark, stinging nettle, hamamelis, hops, camomile, burdock, horsetail, linden blossom, almond, aloe vera, coconut, mango, apricot, lemon, wheat, kiwi, melon, orange, grapefruit, sage, rosemary, birch, lady's smock, wild thyme, yarrow, restharrow, meristem, ginseng and ginger root.

Of very particular suitability for the use according to the invention are the extracts from green tea, almond, aloe vera, coconut, mango, apricot, lemon, wheat, kiwi and melon.

Extractants for producing the specified plant extracts which may be used are water, alcohols and mixtures thereof. Among the alcohols, preference is given here to lower alcohols, such as ethanol and isopropanol, but in particular, polyhydric alcohols, such as ethylene glycol and propylene glycol, both as the sole extractant and also in a mixture with water. Plant extracts based on water/propylene glycol in the ratio 1:10 to 10:1 have proven to be particularly suitable.

According to the invention, the plant extracts can be used either in pure form or in dilute form. If they are used in dilute form, they usually comprise about 2-80% by weight of active substance and, as solvent, the extractant or extractant mixture used during their isolation.

In addition, it may be preferred to use mixtures of two or more, in particular, of two, different plant extracts in the compositions according to the invention.

In addition, it may prove advantageous if penetration auxiliaries and/or swelling agents (M) are present in the compositions according to the invention. These include, for example, urea and urea derivatives, guanidine and derivatives thereof, arginine and derivatives thereof, waterglass, imidazole and derivatives thereof, histidine and derivatives thereof, benzyl alcohol, glycerol, glycol and glycol ethers, propylene glycol and propylene glycol ethers, for example, propylene glycol monoethyl ethers, carbonates, hydrogen carbonates, diols and triols, and in particular, 1,2-diols and 1,3-diols, such as, for example, 1,2-propanediol, 1,2-pentanediol, 1,2-hexanediol, 1,2-dodecanediol, 1,3-propanediol, 1,6-hexanediol, 1,5-pentanediol, 1,4-butanediol.

Advantageously, for the purposes of the invention, short-chain carboxylic acids (N) can additionally assist the active ingredient complex (A). For the purposes of the invention, short-chain carboxylic acids and derivatives thereof are understood as meaning carboxylic acids which may be saturated or unsaturated and/or straight-chain or branched or cyclic and/or aromatic and/or heterocyclic and have a molecular weight of less than 750. For the purposes of the invention, saturated or unsaturated straight-chain or branched carboxylic acids with a chain length of from 1 to 16 carbon atoms in the chain may be preferred, very particular preference being given to those with a chain length of from 1 to 12 carbon atoms in the chain.

For the purposes of the invention, the short-chain carboxylic acids can have one, two, three or more carboxy groups. For the purposes of the invention, preference is given to carboxylic acids with two or more carboxy groups, in particular, di- and tricarboxylic acids. The carboxy groups may be present completely or in part as ester, acid anhydride, lactone, amide, imidic acid, lactam, lactim, dicarboximide, carbohydrazide, hydrazone, hydroxam, hydroxime, amidine, amide oxime, nitrile, phosphonic or phosphate ester. The carboxylic acids according to the invention can of course be substituted along the carbon chain or the ring backbone. The substituents of the carboxylic acids according to the invention are to include, for example, C1-C8-alkyl, C2-C8-alkenyl, aryl, aralkyl and aralkenyl, hydroxymethyl, C2-C8-hydroxyalkyl, C2-C8-hydroxyalkenyl, aminomethyl, C2-C8-aminoalkyl, cyano, formyl, oxo, thioxo, hydroxyl, mercapto, amino, carboxy or imino groups. Preferred substituents are C1-C8-alkyl, hydroxymethyl, hydroxyl, amino and carboxy groups. Particular preference is given to substituents in the □ position. Very particularly preferred substituents are hydroxyl, alkoxy and amino groups, where the amino function may optionally be further substituted by alkyl, aryl, aralkyl and/or alkenyl radicals. Furthermore, likewise preferred carboxylic acid derivatives are the phosphonic and phosphate esters.

Examples of carboxylic acids according to the invention which may be mentioned are formic acid, acetic acid, propionic acid, butyric acid, isobutyric acid, valeric acid, isovaleric acid, pivalic acid, oxalic acid, malonic acid, succinic acid, glutaric acid, glyceric acid, glyoxylic acid, adipic acid, pimelic acid, suberic acid, azelaic acid, sebacic acid, propiolic acid, crotonic acid, isocrotonic acid, elaidic acid, maleic acid, fumaric acid, muconic acid, citraconic acid, mesaconic acid, camphoric acid, benzoic acid, o,m,p-phthalic acid, naphthoic acid, toluoyl acid, hydratropic acid, atropic acid, cinnamic acid, isonicotinic acid, nicotinic acid, bicarbamic acid, 4,4′-dicyano-6,6′-binicotinic acid, 8-carbamoyloctanoic acid, 1,2,4-pentanetricarboxylic acid, 2-pyrrolecarboxylic acid, 1,2,4,6,7-naphthalenepentaacetic acid, malonaldehyde acid, 4-hydroxyphthalamide acid, 1-pyrazolecarboxylic acid, gallic acid or propanetricarboxylic acid, a dicarboxylic acid chosen from the group which is formed by compounds of the general formula (N-I), 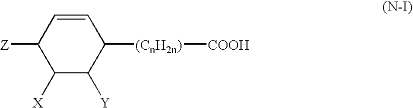
in which Z is a linear or branched alkyl or alkenyl group having 4 to 12 carbon atoms, n is a number from 4 to 12, and one of the two groups X and Y is a COOH group and the other is hydrogen or a methyl or ethyl radical, dicarboxylic acids of the general formula (N-I) which additionally also carry 1 to 3 methyl or ethyl substituents on the cyclohexene ring, and dicarboxylic acids which form from the dicarboxylic acids according to formula (N-I) formally by addition of a molecule of water onto the double bond in the cyclohexene ring.

Dicarboxylic acids of the formula (N-I) are known in the literature.

The dicarboxylic acids of the formula (N-I) can be prepared, for example, by reacting polyunsaturated dicarboxylic acids with unsaturated monocarboxylic acids in the form of a Diels-Alder cyclization. The process usually starts from a polyunsaturated fatty acid as dicarboxylic acid component. Preference is given to the linoleic acid obtainable from natural fats and oils. As monocarboxylic acid component, preference is given in particular, to acrylic acid, but also, for example, methacrylic acid and crotonic acid. Usually, in reactions according to Diels-Alder, isomer mixtures are formed in which one component is present in excess. According to the invention, these isomer mixtures can be used just as much as the pure compounds.

Besides the preferred dicarboxylic acids according to formula (N-I), according to the invention it is also possible to use those dicarboxylic acids which differ from the compounds according to formula (N-I) by 1 to 3 methyl or ethyl substituents on the cyclohexyl ring or are formed from these compounds formally by adding a molecule of water onto the double bond of the cyclohexene ring.

The dicarboxylic acid (mixture) which forms by reacting linoleic acid with acrylic acid has proven particularly advantageous according to the invention. This is a mixture of 5- and 6-carboxy-4-hexyl-2-cyclohexene-1-octanoic acid. Such compounds are commercially available under the names Westvaco Diacid® 1550 and Westvaco Diacid® 1595 (manufacturer: Westvaco).

Besides the short-chain carboxylic acids according to the invention themselves listed above by way of example, it is also possible to use their physiologically compatible salts according to the invention. Examples of such salts are the alkali metal, alkaline earth metal, zinc salts and also ammonium salts, which, for the purposes of the present application, are also understood as meaning the mono-, di- and trimethyl-, -ethyl- and -hydroxyethylammonium salts. However, for the purposes of the invention, very particular preference may be given to using acids neutralized with alkaline-reacting amino acids, such as, for example, arginine, lysine, ornithine and histidine. Furthermore, it may be preferred, for formulation reasons, to choose the carboxylic acid from the water-soluble representatives, in particular, the water-soluble salts.

In addition, it is preferred according to the invention to use hydroxycarboxylic acids and here in turn, in particular, the dihydroxy-, trihydroxy- and polyhydroxycarboxylic acids, and the dihydroxy-, trihydroxy- and polyhydroxy- di-, tri- and polycarboxylic acids together with the active ingredient (A). In this connection, it has been found that, besides the hydroxycarboxylic acids, the hydroxycarboxylic acid esters, and also the mixtures of hydroxycarboxylic acids and esters thereof, and also polymeric hydroxycarboxylic acids and esters thereof may also be very particularly preferred. Preferred hydroxycarboxylic acid esters are, for example, full esters of glycolic acid, lactic acid, malic acid, tartaric acid or citric acid. Further fundamentally suitable hydroxycarboxylic acid esters are esters of β-hydroxy-propionic acid, of tartronic acid, of D-gluconic acid, of sugar acid, of mucic acid or of glucuronic acid. Suitable as alcohol components of these esters are primary, linear or branched aliphatic alcohols having 8-22 carbon atoms, thus, for example, fatty alcohols or synthetic fatty alcohols. Here, the esters of C12-C15-fatty alcohols are particularly preferred. Esters of this type are commercially available, e.g., under the trade name Cosmacol® from EniChem, Augusta Industriale. Particularly preferred polyhydroxypolycarboxylic acids are polylactic acid and polytartaric acid, and esters thereof.

The present invention further provides a method of treating hair comprising contacting the hair with an effective amount of a hair treatment composition according to the invention which results in the improvement of at least one of the following properties
tensile strength of keratin fibers, in particular human hair;
stabilization of the moisture balance of keratin fibers, in particular human hair;
combability of keratin fibers, in particular human hair;
delay in the aging process of keratin fibers, in particular human hair;
restructurability of keratin fibers, in particular human hair, during or after the permanent waving process;
reduction in the decrease in elasticity of keratin fibers, in particular human hair, in the case of damage as a result of atmospheric effects.

With regard to preferred used according to the invention, that stated with regard to preferred compositions according to the invention applies mutatis mutandis.

The examples below are intended to illustrate the subject matter of the invention in more detail without limiting it.

Volume Shampoo.

| | 1 | 2 | 3 |
|---|---|---|---|
| Texapon K 14 S* | 18 | 18 | 18 |
| Citric acid | 0.5 | 0.5 | 0.5 |
| Plantacare 818 UP** | 4 | 4 | 4 |
| Dow Corning 193 surfactant | 0.3 | 0.3 | 0.3 |
| Pantolactone | 0.2 | 0.2 | 0.2 |
| Keratec IFP^{##} | 1.5 | 1.5 | |
| Keratec Pep^{#} | — | 0.5 | 0.5 |
| Sodium chloride | 0.2 | 0.2 | 0.2 |
| Preservative | q.s. | q.s. | q.s. |
| Water | ad 100 | ad 100 | ad 100 |

*Lauryl myristyl ether sulfate sodium salt (about 68% to 73% active substance content; INCI name: Sodium Myreth Sulfate) (Cognis)
**Alkyl polyglucoside (Cognis); INICI: COCO GLUCOSIDE
^{#}Intermediate filament protein from wool, MW = 3 to 4 kDa (Croda)
^{##}Mixture of intermediate filament protein from wool, MW = 40 to 60 kDa and intermediate filament protein from wool, MW = 3 to 4 kDa (Croda)

Care Rinse.

| | 1 | 2 | 3 |
|---|---|---|---|
| Cetearyl alcohol | 5.2 | 5.2 | 5.2 |
| Isopropyl myristate | 1 | 1 | 1 |
| Cutina GMS-V* | 1 | 1 | 1 |
| Dehyquart F 75** | 1 | 1 | 1 |
| Eumulgin B2*** | 0.6 | 0.6 | 0.6 |
| Citric acid | 0.6 | 0.6 | 0.6 |
| Pantolactone | 0.2 | 0.2 | 0.2 |
| Dow Corning 1403 Fluid | 2 | 2 | 2 |
| Dow Corning 949 | 0.2 | 0.2 | 0.2 |
| Keratec IFP^{##} | 1.5 | 1.5 | |
| Keratec Pep^{#} | | 0.5 | 0.5 |
| Preservative | q.s. | q.s. | q.s. |
| Water | ad 100 | ad 100 | ad 100 |

*Glycerol monostearate
**Fatty alcohols/methyltriethanolammonium methylsulfate dialkyl ester mixture (INCI name: Distearoylethyl Hydroxyethylmonium Methosulfate, Cetearyl Alcohol) (COGNIS)
***Cetylstearyl alcohol, ethoxylated with 20 mol of EO
^{#}Intermediate filament protein from wool, MW = 3 to 4 KDa (Croda)
^{##}Mixture of intermediate filament protein from wool, MW = 40 to 60 kDa and intermediate filament protein from wool MW = 3 to 4 kDa (Croda)

Hair Ends Fluids.

| | 1 | 2 | 3 |
|---|---|---|---|
| Dow Corning 1403 Fluid | 29 | 29 | 29 |
| Dow Corning 9040 | 25 | 25 | 25 |
| Trisiloxane Fluid 1 cs | 44 | 43.5 | 45 |
| Vitamin E | 0.5 | 0.5 | 0.5 |
| Keratec IFP^{##} | 1.5 | 1.5 | — |
| Keratec Pep^{#} | — | 0.5 | 0.5 |

^{#}Intermediate filament protein from wool, MW = 3 to 4 kDa (Croda)
^{##}Mixture of intermediate filament protein from wool, MW = 40 to 60 kDa and intermediate filament protein from wool, MW = 3 to 4 kDa (Croda)

Leave-On Treatment.

| | |
|---|---|
| Lactic acid | 0.08 |
| Panthenol | 0.4 |
| Dow Corning 5330 Fluid | 2 |
| Dow Corning 949 | 5 |
| Ethanol | 10 |
| Water | ad 100 |
| Keratec Pep^{#} | 0.5 |
| Preservative | q.s. |

^{#}Intermediate filament protein from wool, MW = 3 to 4 kDa (Croda)

1. A corneocyte protein- or polypeptide-containing hair-treatment composition, comprising
a) from 0.01 to 5% by weight of at least one corneocyte protein or polypeptide;
b) from 0.05 to 95% by weight of at least one silicone.

2. The hair-treatment composition of claim 1 wherein the corneocyte protein or polypeptide has a molar mass of from 20 to 80 kDa.

3. The hair-treatment composition of claim 2 wherein the molar mass is from 45 to 55 kDa.

4. The hair-treatment composition of claim 1 wherein the corenocyte protein or polypeptide has a molar mass of from 2 to 8 kDa.

5. The hair-treatment composition of claim 4 wherein the molar mass is from 3 to 4.5 kDa.

6. The hair-treatment composition of claim 1 wherein component a) is comprised of i) a corneocyte protein or polypeptide having a molar mass of from 20 to 80 kDa and ii) a corenocyte protein or polypeptide having a molar mass of from 2 to 8 kDa wherein the weight ratio of i)/ii) is from 100:1 to 1:100.

7. The hair-treatment composition of claim 6 wherein the ratio is from 5:1 to 1:2.

8. The hair-treatment composition of claim 1 wherein the silicone of component b) is a compound of the formula I
(CH₃)₃—Si—[O—Si(CH₃)₂]ₓ—O—Si(CH₃)₃ (1)
wherein x is a number from 0 to 100.

9. The hair-treatment composition of claim 8, wherein x is a number from 0 to 10.

10. The hair-treatment composition of claim 1 wherein the silicone of component b) is an amino-functional silicone of the formula (II)
R′ₐG₃₋ₐ-Si(OSiG₂)ₙ-(OSiG_{b}R′_{2-b})ₘ—O—SiG₃₋ₐ-R′ₐ (II),
wherein
G is —H, a phenyl group, —OH, —O—CH₃, —CH₃, —O—CH₂CH₃, —CH₂CH₃, —O—CH₂CH₂CH₃, —CH₂CH₂CH₃, —O—CH(CH₃)₂, —CH(CH₃)₂, —O—CH₂CH₂CH₂CH₃, —CH₂CH₂CH₂CH₃, —O—CH₂CH(CH₃)₂, —CH₂CH(CH₃)₂, —O—CH(CH₃)CH₂CH₃, —CH(CH₃)CH₂CH₃, —O—C(CH₃)₃, —C(CH₃)₃;
a is a number between 0 and 3, in particular, 0;
b is a number between 0 and 1, in particular, 1,
m and n are numbers whose sum (m+n) is between 1 and 2,000, preferably between 50 and 150, where n preferably assumes values from 0 to 1,999 and in particular, from 49 to 149 and m preferably assumes values from 1 to 2,000, in particular, from 1 to 10,
R′ is a monovalent radical selected from
-QN(R″)—CH₂—CH₂—N(R″)₂
-Q-N(R″)₂
-Q-N⁺(R″)₃A⁻
-Q-N⁺H(R″)₂A⁻
-Q-N⁺H₂(R″)A⁻
-Q-N(R″)—CH₂—CH₂—N⁺R″H₂A⁻,
where each Q is a chemical bond, —CH₂—, —CH₂—CH₂—, —CH₂CH₂CH₂—, —C(CH₃)₂—, —CH₂CH₂CH₂CH₂—, —CH₂C(CH₃)₂—, —CH(CH₃)CH₂CH₂—,
R″ is identical or different radicals from the group —H, -phenyl, -benzyl, —CH₂—CH(CH₃)Ph, the C₁₋₂₀-alkyl radicals, preferably —CH₃, —CH₂CH₃, —CH₂CH₂CH₃, —CH(CH₃)₂, —CH₂CH₂CH₂H₃, —CH₂CH(CH₃)₂, —CH(CH₃)CH₂CH₃, —C(CH₃)₃, and A represents an anion which is preferably selected from chloride, bromide, iodide or methosulfate.

11. The hair-treatment composition of claim 1 wherein the silicone of component b) is a compound of the formula III 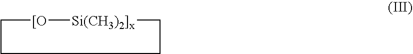
wherein x is a number from 3 to 200.

12. The hair-treatment composition of claim 11 wherein x is a number from 3 to 7.

13. The hair-treatment composition of claim 1 wherein the silicone component b) is a compound of the formula IV
R₃SI—[O—SiR₂]x-(CH₂)ₙ—[O—Sir₂]y—O—SiR₃ (IV)
wherein each of R₂ and R₃ is independently —H, -phenyl, -benzyl, —CH₂—CH(CH₃)Ph, a C₁₋₂₀ alkyl radical and each of x and y is independently a number from 0 to 200.

14. The hair-treatment composition of claim 13 wherein each of x and y is independently a number from 1 to 8.

15. The hair-treatment composition of claim 1 wherein the silicone of component b) is a water-soluble silicone.

16. The hair-treatment composition of claim 1 further comprising from 0.5 to 70% by weight of an anionic surfactant, a nonionic surfactant, a cationic surfactant, an amphoteric surfactant or a combination thereof.

17. The hair-treatment composition of claim 16 wherein the amount of the anionic surfactant, a nonionic surfactant, a cationic surfactant, an amphoteric surfactant or a combination thereof is from 5 to 25% by weight.

18. The hair-treatment composition of claim 1 further comprising from 0.1 to 5% by weight of vitamins, provitamins and vitamin precursors from the vitamin groups A, B, C, E, F and H.

19. The hair treatment composition of claim 18 wherein the amount of the vitamins, provitamins and vitamin precursors from the vitamin groups A, B, C, E, F and H is from 0.5 to 25% by weight.

20. A method of treating hair comprising contacting the hair with an effective amount of a hair treatment composition according to the invention which results in the improvement of at least one of the following properties
tensile strength of keratin fibers;
stabilization of the moisture balance of keratin fibers;
combability of keratin fibers;
delay in the aging process of keratin fibers;
restructurability of keratin fibers;
reduction in the decrease in elasticity of keratin fibers, in particular human hair, in the case of damage as a result of atmospheric effects.
